(19) United States
(12) Patent Application Publication
(10) Pub. No.: US 2020/0075142 A1
(43) Pub. Date: Mar. 5, 2020

(54) MEDICAL INFORMATION DISPLAY APPARATUS

(71) Applicant: Canon Medical Systems Corporation, Otawara-shi (JP)

(72) Inventors: Yusuke Kano, Nasushiobara (JP); Shinya Sugiyama, Nasushiobara (JP); Kazuki Utsunomiya, Nasushiobara (JP); Longxun Piao, Nasushiobara (JP); Anri Sato, Nasushiobara (JP)

(73) Assignee: Canon Medical Systems Corporation, Otawara-shi (JP)

(21) Appl. No.: 16/551,802

(22) Filed: Aug. 27, 2019

(30) Foreign Application Priority Data
Aug. 29, 2018 (JP) 2018-160725
Aug. 23, 2019 (JP) 2019-153123

(51) Int. Cl. G16H 10/60; G16H 20/00; G16H 30/20

(52) U.S. Cl. CPC G16H 10/60; G16H 30/20; G16H 20/00

(57) ABSTRACT

According to one embodiment, a medical information display apparatus includes processing circuitry. The processing circuitry acquires patient identification information for identifying a patient in whom an event as a determination index in a medical treatment has occurred and time information representing a time associated with the event from medical treatment information of a plurality of patients, generates mapping data by mapping one or more icons associated with the event along a common time axis based on the plurality of pieces of patient identification information and the plurality of pieces of time information so as to make the icons identifiable for each patient, and displays the mapping data on a display.

### CROSS-REFERENCE TO RELATED APPLICATIONS

This application is based upon and claims the benefit of priority from Japanese Patent Applications No. 2018-160725, filed Aug. 29, 2018; and No. 2019-153123, filed Aug. 23, 2019; the entire contents of both of which are incorporated herein by reference.

### FIELD

Embodiments described herein relate generally to a medical information display apparatus.

### BACKGROUND

Recently, there has been available a medical information display apparatus equipped with a medical treatment information integrated viewer that enables integrated observation of medical treatment information about a patient. Such a medical information display apparatus allows a user such as a doctor to integrally observe medical treatment information about a patient which is displayed on the display of the medical information display apparatus and perform diagnosis, therapy, and the like of the patient. The medical treatment information about the patient includes, for example, various types of information such as medical image data representing a medical image and electronic health record data. More specifically, the medical treatment information includes, for example, items such as examination results of image examination, specimen examination, bacteria examination, and the like, information concerning vital sign, medicine administration, and the like, diagnostic reading report, and medical record description contents. Medical treatment information about patients is daily stored in a predetermined database.

When using a medical information display apparatus implementing such an integrated viewer, for example, the user can arbitrarily select items of medical treatment information on a screen displayed on the display, arrange the selected items in a predetermined area, and display the corresponding information.

In order to improve the efficiency of medical treatment services performed by each doctor, it is important to quickly access medical treatment information about a patient to be preferentially treated. In this case, according to the prior art, for example, information representing the progresses of medical treatment for patients is displayed for each patient. In this case, the user determines the priority levels of checks on medical treatment information about patients among the patients while checking information representing the progress of medical treatment for each patient. Accordingly, it takes much time to access medical treatment information about a patient to be preferentially treated.

### BRIEF DESCRIPTION OF THE DRAWINGS

FIG. 1 is a block diagram representing a medical treatment information system including a medical information display apparatus according to an embodiment;

FIG. 2 is a block diagram showing the functional arrangement of the medical information display apparatus shown in FIG. 1;

FIG. 3 is a flowchart representing an example of the operation of the processing circuitry of the medical information display apparatus according to the embodiment;

FIG. 4 is a view showing an example of a medical treatment screen concerning cardiac failure sign event displayed on a display shown in FIG. 2;

FIG. 5 is a view showing an example of a medical treatment information screen concerning surgery schedule events displayed on the display shown in FIG. 2;

FIG. 6 is a view showing an example of a medical treatment screen concerning image examination events displayed on the display shown in FIG. 2;

FIG. 7 is a view showing a display example of mapping data according to the first modification;

FIG. 8 is a view showing another display example of mapping data according to the first modification;

FIG. 9 is a view showing still another display example of mapping data according to the first modification;

FIG. 10 is a view showing a display example of mapping data according to the second modification;

FIG. 11 is a view showing another display example of mapping data according to the second modification;

FIG. 12 is a view showing still another display example of mapping data according to the according to the second modification;

FIG. 13 is a view showing still another display example of mapping data according to the according to the second modification;

FIG. 14 is a view showing a display example of mapping data according to the third modification;

FIG. 15 is a view showing another display example of mapping data according to the third modification;

FIG. 16 is a view showing still another display example of mapping data according to the third modification;

FIG. 17 is a view showing a display example of mapping data according to the fourth modification;

FIG. 18 is a view showing another display example of mapping data according to the fourth modification;

FIG. 19 is a view showing still another display example of mapping data according to the fourth modification;

FIG. 20 is a view representing an example of a medical treatment screen according to another embodiment; and

FIG. 21 is a view representing an example of a patent detail screen representing the details of medical treatment information about a patient according to another embodiment.

### DETAILED DESCRIPTION

In general, according to one embodiment, a medical information display apparatus includes processing circuitry. The processing circuitry acquires patient identification information for identifying a patient in whom an event as a determination index in a medical treatment has occurred and time information representing a time associated with the event from medical treatment information of a plurality of patients, generates mapping data by mapping one or more icons associated with the events along a common time axis based on the plurality of pieces of patient identification information and the plurality of pieces of time information so as to make the icons identifiable for each patient, and displays the mapping data on a display.

Embodiments will be described below with reference to the accompanying drawings.

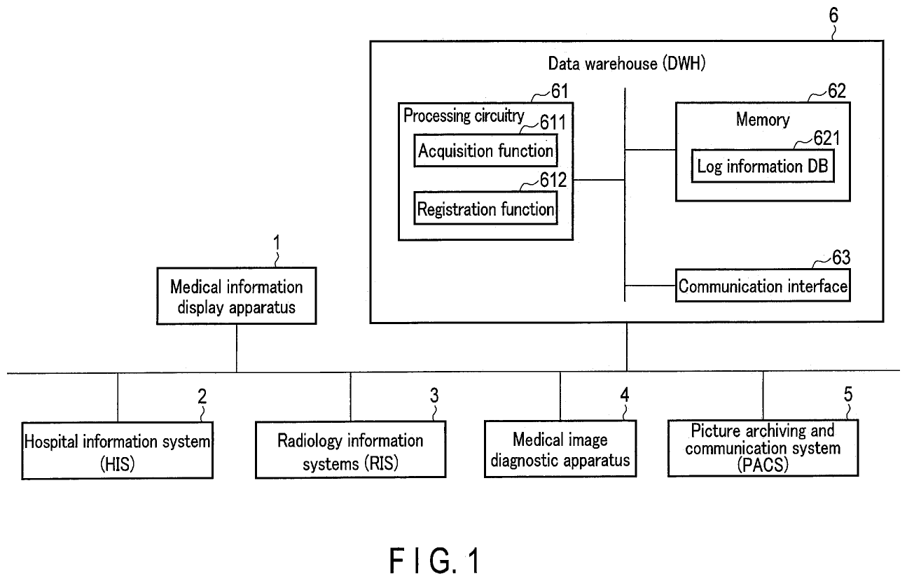

FIG. 1 is a block diagram showing an example of an environment used by a medical information display apparatus 1 according to this embodiment. The medical information display apparatus 1 shown in FIG. 1 is, for example, an apparatus that enables integrated observation of medical treatment information. The medical information display apparatus 1 implements, for example, an integrated viewer. The integrated viewer is an application for integrally presenting medical treatment information to the user. The integrated viewer may use any of the following implementation forms such as a Web application, a fat client application, and a thin client application. The medical information display apparatus 1 is communicably connected to an HIS (Hospital Information System) 2, a RIS (Radiology Information Systems) 3, a medical image diagnosis apparatus 4, a PACS (Picture Archiving and Communication System) 5, and a DWH (Data WareHouse) 6 via an intra-hospital network such as a LAN (Local Area Network).

Referring to FIG. 1, the HIS 2 includes an electronic health record system that manages information concerning electronic health records. Information concerning an electronic health record includes, for example, patient information and medical treatment information. Patient information is information unique to a patient, and includes, for example, a patient ID, patient name, sex, and age.

Medical treatment information is information that can be known by a medical worker, such as the physical status of a patient, a medical condition, and medical treatment, in the process of medical treatment. Medical treatment information includes, for example, image information, examination history information, electrocardiogram information, vital sign information, medication history information, report information, medical chart description information, and nursing record information. Image information is information representing the location of a medical image obtained by imaging a patient. Image information includes, for example, information representing the location of a medical image file (to be described later) generated by the medical image diagnosis apparatus 4 that has executed an examination. Examination history information is information representing, for example, the history of examination results obtained by performing specimen examination, bacteria examination, and the like with respect to a patient. Electrocardiogram information is information concerning the electrocardiographic waveform measured from a patient. Vital sign information is basic information concerning the life of a patient. Vital sign information includes, for example, a pulse rate, respiration rate, oxygen concentration, body temperature, blood pressure, and level of consciousness. Medication history information is information representing, for example, the history of the amounts of medicines administered to a patient. Report information is information obtained by a reading doctor in a radiology department by diagnostically reading medical images such as X-ray images, CT images, MRI images, and ultrasonic images in response to an examination request from a medical treatment doctor in a diagnosis and treatment department and summarizing information about the condition and disease of the patient. Report information includes, for example, reading report information representing the reading report generated by a reading doctor by referring to a medical image file stored in the PACS 5. Note that report information is generally stored in the PACS 5, and hence the electronic health record system can display report information by reading out the report information from the PACS 5.

Medical record description information is, for example, information input to an electronic health record by a medical treatment doctor or the like. Medical record description information includes, for example, a medical treatment record at hospital admission, the medical history of a patient, and a medication prescription history.

Nursing record information is, for example, information input to an electronic health record by a nurse or the like. Nursing record information includes a nursing record at hospital admission.

Information concerning an electronic health record includes, for example, examination execution information. Examination execution information is generated by the medical image diagnosis apparatus 4, which has executed an examination according to examination order information. Examination execution information is information representing the examination executed by the medical image diagnosis apparatus 4. Examination execution information includes an order number, examination UID (Unique ID), patient ID, modality type, imaging region, and imaging conditions. An examination UID is an identifier that can uniquely specify an examination. A modality type represents the modality used for imaging. Modality types include, for example, “X-ray computed tomography apparatus”, “X-ray diagnosis apparatus”, “magnetic resonance imaging apparatus”, and “ultrasonic diagnosis apparatus”. An imaging region corresponds to an examination region included in examination order information. Imaging regions include, for example, abdomen, brain, and breast. Imaging conditions include a body posture, imaging direction, and whether to use a contrast medium.

The HIS 2 includes an order system that manages reservation information, order information, and the like. Note that the HIS 2 is configured to include an electronic health record system and an ordering system.

Reservation information includes, for example, information concerning a clinical consultation reservation, examination reservation, and the like. Information concerning a clinical consultation reservation includes, for example, a clinical consultation day, clinical consultation time, receipt number, doctor for clinical consultation, and department for clinical consultation. Information concerning an examination reservation includes, for example, an examination day, examination time, and receipt number. Order information is, for example, information concerning an order requested by a clinical consultation doctor or the like, and is, for example, order information concerning an image examination, specimen examination, physiological examination, prescription, and medicine administration. When order information is examination order information for requesting an image examination, the examination order information includes, for example, an order number that can identify an examination, patient ID, examination type, examination region, and request source information. An order number is a number issued when examination order information is input, and is, for example, an identifier for uniquely specifying examination order information in one hospital. Examination types include an X-ray examination, CT (computed Tomography) examination, MR (Magnetic Resonance) examination, and RI (Radio Isotope) examination. Examination regions include, for example, abdomen, brain, and breast. Request source information includes a clinical consultation department name and the name of a doctor in charge. Information concerning an examination reservation is linked to order information.

The RIS 3 is a system that manages examination reservation information associated with a radiographic examination service. For example, the RIS 3 collects examination order information input from clinical consultation doctors in an order system included in the HIS 2 upon adding various types of setting information to the examination order information, and manages the collected information as examination reservation information. Note that the RIS 3 may add various types of setting information to examination order information by using radiation records recording various types of setting information set in the medical image diagnosis apparatus 4 in past examinations. The RIS 3 transmits an examination order to the medical image diagnosis apparatus 4 according to examination reservation information. The RIS 3 also transmits the examination execution information generated by the medical image diagnosis apparatus 4 used to execute an examination to an electronic health record system included in the HIS 2.

The medical image diagnosis apparatus 4 is an apparatus that executes an examination by, for example, imaging a patient. The medical image diagnosis apparatus 4 includes, for example, an X-ray computed tomography apparatus, X-ray diagnosis apparatus, magnetic resonance imaging apparatus, nuclear medicine diagnosis apparatus, and ultrasonic diagnosis apparatus. The medical image diagnosis apparatus 4 executes an examination based on, for example, the examination reservation information transmitted from the RIS 3. The medical image diagnosis apparatus 4 generates examination execution information and transmits it to the RIS 3.

The medical image diagnosis apparatus 4 generates medical image data by executing an examination. Medical image data is, for example, X-ray CT image data, X-ray image data, MRI image data, nuclear medicine image data, or ultrasonic image data. The medical image diagnosis apparatus 4 generates a medical image file by converting generated medical image data into a form complying with the DICOM (Digital Imaging and Communication in Medicine) standards. A medical image file is, for example, a file in a form complying with the DICOM standards. The medical image diagnosis apparatus 4 transmits the generated medical image file to the PACS 5.

The PACS 5 is a system that manages various types of medical image files. The PACS 5 stores, for example, the medical image file transmitted from the medical image diagnosis apparatus 4. Note that the PACS 5 may store report information attached to a medical image file or report information about examinations associated with a plurality of medical image files.

The DWH 6 is a database system that collectively accumulates, for example, information generated by healthcare providers, that is, medical treatment big data. The DWH 6 is implemented by, for example, a general server apparatus. For example, as shown in FIG. 1, the DWH 6 includes processing circuitry 61, a memory 62, and a communication interface 63. The processing circuitry 61, the memory 62, and the communication interface 63 are communicably connected to each other via, for example, a bus.

The processing circuitry 61 is a processor that functions as the main unit of the DWH 6. The processing circuitry 61 executes programs stored in the memory 62 or the like to implement, for example, an acquisition function 611 and a registration function 612 corresponding to the programs.

The acquisition function 611 is a function of acquiring desired information from the HIS 2, the RIS 3, the medical image diagnosis apparatus 4, and the PACS 5. More specifically, with the acquisition function 611, the processing circuitry 61 acquires, for example, preset information in a preset cycle from the HIS 2, the RIS 3, the medical image diagnosis apparatus 4, and the PACS 5. Note that the processing circuitry 61 may acquire desired information from the HIS 2, the RIS 3, the medical image diagnosis apparatus 4, and the PACS 5 by using a machine learning model stored in advance. With this operation, the processing circuitry 61 acquires information associated with an electronic health record and the like from the HIS 2, examination reservation information and the like from the RIS 3, and medical image files from the medical image diagnosis apparatus 4 or the PACS 5.

Note that the processing circuitry 61 may acquire first various types of information from the HIS 2, the RIS 3, the medical image diagnosis apparatus 4, and the PACS 5 and then acquire desired information from the acquired information.

The registration function 612 is a function of causing the memory 62 to store the information acquired by the acquisition function 611. More specifically, with the registration function 612, the processing circuitry 61 causes the memory 62 to store, for example, the medical treatment information acquired from the HIS 2 according to a preset rule. The preset rule is, for example, a sequence using event dates and the like associated with medical treatment events for each patient, such as outpatient consultation, surgery, image examination, specimen examination, bacteria examination, electrocardiographic measurement, vital sign measurement, medicine administration, report creation, and medical record description. An event date is, for example, a date at which a medical treatment event has occurred or a date at which a medical treatment event is scheduled. Event dates include, for example, the execution date of an image examination, the execution date of a specimen examination, the measurement date of an electrocardiographic waveform, the measurement date of a vital sign, the date of administration of a medicine, the creation date of a report, and the date of description of a medical record. The medical treatment information acquired in this manner is stored as log information 621 in the memory 62.

The memory 62 is a storage device such as an HDD (hard disk drive), SSD (solid state drive), or integrated circuit storage device that stores various types of information. The memory 62 may be a driving device that reads and writes various types of information from and to a portable storage medium such as a CD-ROM drive, DVD drive, or flash memory. The memory 62 stores, for example, control programs and the like for implementing the acquisition function 611 and the registration function 612. Note that the programs may be distributed to, for example, nonvolatile storage media and read out from the nonvolatile storage media and installed in the memory 62. The memory 62 stores, for example, the log information 621.

The communication interface 63 performs data communication among the medical information display apparatus 1, the HIS 2, the RIS 3, the medical image diagnosis apparatus 4, and the PACS 5 via an intra-hospital network. Any communication standards may be used among the medical information display apparatus 1, the HIS 2, the RIS 3, the medical image diagnosis apparatus 4, and the PACS 5. For example, it is possible to use, for example, the HL7 (Health Level 7), the DICOM, or the both of them.

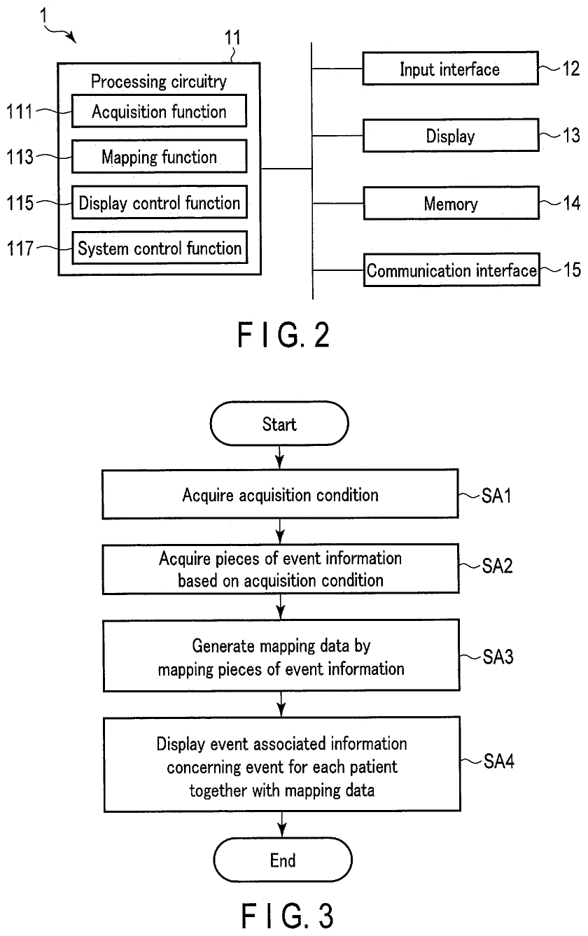

The medical information display apparatus 1 according to this embodiment will be described in detail with reference to FIG. 2. FIG. 2 is a block diagram showing the functional arrangement of the medical information display apparatus 1 shown in FIG. 1.

The medical information display apparatus 1 shown in FIG. 2 includes processing circuitry 11, an input interface 12, a display 13, a memory 14, and a communication interface 15. The processing circuitry 11, the input interface 12, the display 13, the memory 14, and the communication interface 15 are communicably connected to each other via, for example, a bus.

The processing circuitry 11 is a processor functioning as the main unit of the medical information display apparatus 1. The medical information display apparatus 1 executes a control program stored in the memory 14 or the like to implement a function corresponding to the program.

The input interface 12 is implemented by, for example, a mouse, a keyboard, and a touch panel having an operation screen which the operator touches to input an instruction. The input interface 12 accepts, for example, a display instruction from the operator. The input interface 12 converts a display instruction from the operator into an electrical signal and outputs the electrical signal to the processing circuitry. A display instruction is, for example, to designate a button displayed on the screen and to designate a character string from a pull-down menu.

The display 13 displays various types of information for the execution of various types of services by the user. As the display 13, for example, a CRT display, liquid crystal display, organic EL display, LED display, plasma display, or another arbitrary display known in the art can be used, as appropriate.

The memory 14 is a storage device such as an HDD (Hard Disk Drive), SSD (Solid State Drive), or integrated circuit storage device, which stores various types of information. In addition, the memory 14 may be a driving device or the like that reads and writes various types of information between portable storage media such as a CD-ROM drive, DVD drive, and flash memory.

The memory 14 stores an acquisition condition for the acquisition of event information associated with an event as an index for determination in a medical treatment. Even information is, for example, information representing an arbitrary record associated with a medical treatment for a patient. Event information includes at least one of the respective items for medical treatment information. Note that event information includes, for example, a predetermined character string included in medical treatment information, a result of analysis on numerical values and the like, or a fact that is determined to be notified (warned) to the user. Events include an event in a medical treatment workflow specified in advance concerning a medical treatment service and an event detected from the medical treatment information of a patient based on an empirical rule concerning a medical treatment.

Acquisition conditions are based on, for example, predetermined rules. The predetermined rules include, for example, a rule specified by a medical treatment event occurring in a hospital. The predetermined rules include, for example, a rule specified by a public guideline. The predetermined rules include, for example, a rule set by medical facilities or an individual doctor. Acquisition conditions include, for example, an event type representing the type of event.

Acquisition conditions may be acquisition conditions specified based on AI (Artificial Intelligence) techniques and the like.

The communication interface 15 performs data communication with the HIS 2, the RIS 3, the medical image diagnosis apparatus 4, the PACS 5, and the DWH 6 that are connected to each other via an intra-hospital network. Any communication standards can be used for the HIS 2, the RIS 3, the medical image diagnosis apparatus 4, the PACS 5, and the DWH 6. For example, either or both of HL7 and DICOM can be used.

The processing circuitry 11 according to this embodiment includes an acquisition function 111, a mapping function 113, a display control function 115, and a system control function 117.

The acquisition function 111 is a function of acquiring, from medical treatment information about a plurality of patients, patient identification information for identifying a patient corresponding to the occurrence of an event as an index for determination in a medical treatment and time information representing the time associated with the event. Time information is represented by, for example, a year, a month, a date, or a date and time. The processing circuitry 11 executes the acquisition function 111 to read out an acquisition condition stored in the memory 14 based on an input via the input interface 12 and acquire a plurality of event information concerning events for each patient from the log information 621 stored in the memory 62 of the DWH 6. Each record included in each of a plurality of pieces of acquired event information includes, as items, for example, an event type, patient ID (patient identification information), event date (time information) associated with the event represented by the event information, and event associated information associated with the event. Note that, the patient identification information may include patient name.

The mapping function 113 is a function of mapping event information. The processing circuitry 11 executes the mapping function 113 to generate mapping data by mapping one or more icons concerning events along a common time axis so as to make the icons identifiable for each patient based on the plurality of pieces of patient identification information and the plurality of pieces of time information acquired by the acquisition function 111. As an icon concerning an event, for example, a patient name or a patient ID for specifying the patient corresponding to the event may be displayed. In addition, an icon concerning an event, an event name for specifying the event may be displayed. Note that a symbol mapped to mapping data is not limited to an icon and may be a symbol that can specify an event by its shape.

The display control function 115 is a function of displaying mapping data on the display. The processing circuitry 11 executes the display control function 115 to display, for example, the mapping data generated by the mapping function 113 on the display 13. In this case, the processing circuitry 11 may display event associated information associated with an event for each patient together with mapping data.

The system control function 117 is a function of controlling the basic operations of the medical information display apparatus 1, such as an input/output operation and a communication operation. The processing circuitry 11 executes the system control function 117 to receive various types of requests via the input interface 12. The processing circuitry 11 executes various types of functions in accordance with various types of received requests.

The acquisition function 111, the mapping function 113, the display control function 115, and the system control function 117 may be incorporated as control programs or dedicated hardware circuits that can execute the respective functions may be incorporated in the processing circuitry 11.

Various types of operations of the medical information display apparatus 1 according to this embodiment will be described next with reference to the accompanying drawings.

FIG. 3 is a flowchart showing an example of the operation of the processing circuitry 11 of the medical information display apparatus 1 according to this embodiment. Assume that in the following description, the processing circuitry 11 receives the designation of an event type via the input interface 12. Assume that in this case, the processing circuitry 11 displays, for example, character strings representing event type candidates in a pull-down form on the display 13, and receives the designation of an event type. Assume that the processing circuitry 11 receives the designation of event type “cardiac failure sign event”. Assume also that acquisition conditions for acquiring event information (cardiac failure sign event information) representing this cardiac failure sign event include, for example, “an element concerning biological information acquired from a patient”. Assume that elements concerning biological information acquired from a patient include, for example, a condition concerning a urine output “a daily urine output should be 400 ml or less”. Assume also that the acquisition conditions include a temporal element (for example, a period) and elements representing attributes of a patient (for example, a doctor in charge, medical treatment department, and hospital ward). A temporal element is, for example, “the period from the event date of Apr. 10, 2017 to the event date of Apr. 27, 2017”. An element representing an attribute of a patient is, for example, a patient attended by doctor A. In this case, patients attended by doctor A correspond to patient IDs “00000001”, “00000002”, “00000003”, “00000004”, and “00000005”.

Assume that with regard to the patient with patient ID “00000001”, the dates that satisfy acquisition condition “a daily urine output should be 400 ml or less” are “Apr. 24, 2017”, “Apr. 25, 2017”, and “Apr. 26, 2017”, and the urine outputs obtained at the respective dates are “400 ml”, “300 ml”, and “400 ml”. Assume also that with regard to the patient with patient ID “00000002”, the date that satisfies acquisition condition “a daily urine output should be 400 ml or less” is “Apr. 15, 2017”, and the urine output obtained at the date is “400 ml”. Assume also that with regard to the patient with patient ID “00000003”, the date that satisfies acquisition condition “a daily urine output should be 400 ml or less” is “Apr. 10, 2017”, and the urine output obtained at the date is “400 ml”. Assume also that with regard to the patient with patient ID “00000004”, the date that satisfies acquisition condition “a daily urine output should be 400 ml or less” is “Apr. 14, 2017”, and the urine output obtained at the date is “400 ml”. Assume also that with regard to the patient with patient ID “00000005”, the date that satisfies acquisition condition “a daily urine output should be 400 ml or less” is “Apr. 18, 2017”, and the urine output obtained at the date is “400 ml”.

Note that elements concerning biological information acquired from a patient, which are included in acquisition conditions concerning a cardiac failure sign event, may include, for example, condition “an increase in body weight should be 3 kg or more in two days” concerning a change in body weight or condition “BNP (Brain Natriuretic Peptide) should be 200 pg/ml or more” concerning blood components in addition to condition “a daily urine output should be 400 ml or less” concerning an urine output. In addition, acquisition conditions concerning a cardiac failure sign event may include conditions concerning the description contents of nursing records and the like.

In addition, an event type is not limited to “cardiac failure sign event”. Event types include, for example, “renal function deterioration event”. In this case, elements concerning biological information acquired from a patient, which are included in acquisition conditions concerning a renal function deterioration event, include, for example, condition “a serum creatinine increase within 48 hours should be 0.3 mg/dl or more” concerning blood components.

Upon receiving the designation of an acquisition condition, the processing circuitry 11 reads out the designated acquisition condition from the memory 14 (step SA1). The processing circuitry 11 reads out, for example, an acquisition condition for acquiring a cardiac failure sign event from the memory 14.

The processing circuitry 11 acquires a plurality of pieces of event information based on the readout acquisition condition (step SA2). The processing circuitry 11 acquires, for example, cardiac failure sign event information for each patient from information representing daily urine output records included in the log information 621 stored in the memory 62 of the DWH 6 based on the acquisition condition for acquiring the readout cardiac failure sign event. In other words, the processing circuitry 11 acquires patient identification information for identifying a patient who has experienced cardiac failure sign event and temporal information representing the times associated with the cardiac failure sign event.

More specifically, the processing circuitry 11 determines that the patient with patient ID “00000001” has experienced cardiac failure sign event, because the urine outputs of the patient obtained on the dates “Apr. 24, 2017”, “Apr. 25, 2017”, and “Apr. 26, 2017” are respectively “400 ml”, “300 ml”, and “400 ml”, and acquires necessary information from the log information 621. The processing circuitry 11 also determines that the patient with patient ID “00000002” has experienced a cardiac failure sign event, because the urine output of the patient obtained on the date “Apr. 15, 2017” is “400 ml”, and acquires necessary information from the log information 621. The processing circuitry 11 also determines that the patient with patient ID “00000003” has experienced a cardiac failure sign event, because the urine output of the patient obtained on the date “Apr. 10, 2017” is “400 ml”, and acquires necessary information from the log information 621. The processing circuitry 11 also determines that the patient with patient ID “00000004” has experienced a cardiac failure sign event, because the urine output of the patient obtained on the date “Apr. 14 2017” is “400 ml”, and acquires necessary information from the log information 621. The processing circuitry 11 also determines that the patient with patient ID “00000005” has experienced a cardiac failure sign event, because the urine output of the patient obtained on the date “Apr. 18 2017” is “400 ml”, and acquires necessary information from the log information 621.

In this case, the acquired cardiac failure sign event information includes a record having event type “cardiac failure sign event”, patient ID “00000001”, event date “Apr. 24, 2017”, event associated information (cardiac failure sign event associated information) associated with cardiac failure sign event, and the like. The cardiac failure sign event associated information is, for example, information representing a daily urine output shift in a predetermined period, which is obtained from the patient with patient ID “00000001”. In addition, the cardiac failure sign event information includes a record having event type “cardiac failure sign event”, patient ID “00000001”, event date “Apr. 25, 2017”, and cardiac failure sign event associated information. The cardiac failure sign event information also includes a record having event type “cardiac failure sign event”, patient ID “00000001”, event date “Apr. 26, 2017”, and cardiac failure sign event associated information. Note that the contents of the cardiac failure sign event associated information of the three records with patient ID “00000001” are the same.

The cardiac failure sign event information includes a record having event type “cardiac failure sign event”, patient ID “00000002”, event date “Apr. 15, 2017”, and cardiac failure sign event associated information. The cardiac failure sign event associated information is, for example, information representing a daily urine output shift in a predetermined period, which is obtained from the patient with patient ID “00000002”. The cardiac failure sign event information includes a record having event type “cardiac failure sign event”, patient ID “00000003”, event date “Apr. 10, 2017”, and cardiac failure sign event associated information. The cardiac failure sign event associated information is, for example, information representing a daily urine output shift in a predetermined period, which is obtained from the patient with patient ID “00000003”.

The cardiac failure sign event information includes a record having event type “cardiac failure sign event”, patient ID “00000004”, event date “Apr. 14, 2017”, and cardiac failure sign event associated information. The cardiac failure sign event associated information is, for example, information representing a daily urine output shift in a predetermined period, which is obtained from the patient with patient ID “00000004”. The cardiac failure sign event information includes a record having event type “cardiac failure sign event”, patient ID “00000005”, event date “Apr. 18, 2017”, and cardiac failure sign event associated information. The cardiac failure sign event associated information is, for example, information representing a daily urine output shift in a predetermined period, which is obtained from the patient with patient ID “00000005”.

The processing circuitry 11 maps the plurality of pieces of event information acquired from the plurality of patients (step SA3). More specifically, the processing circuitry 11 generates mapping data by mapping one or more icons concerning cardiac failure sign event along a common time axis so as to make them identifiable for each patient based on the plurality of pieces of acquired patient identification information and the plurality of pieces of acquired time information.

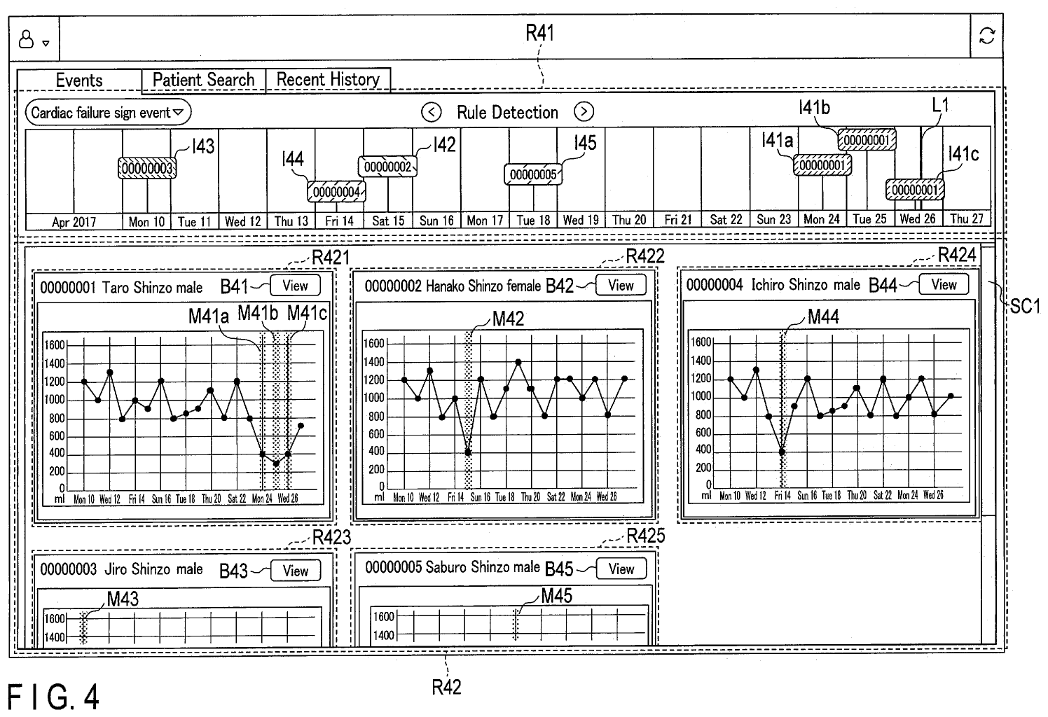

The processing circuitry 11 displays event associated information associated with events for each patient together with the mapping data (step SA4). FIG. 4 is a view showing an example of a medical treatment screen concerning cardiac failure sign event displayed on the display 13 shown in FIG. 2. The medical treatment screen shown in FIG. 4 includes a mapping area R41 having mapping data and an event associated information area R42 having at least one piece of event associated information.

The mapping area R41 includes icons I41a, I41b, I41c, I42, I43, I44, and I45 representing the respective patients. As shown in FIG. 4, the respective icons included in the mapping area R41 are displayed in different colors for each patient. Note that, the respective icons may display with patient name.

The icon I41a represents the fact that the patient with patient ID “00000001” has experienced a cardiac failure sign event on event date “Apr. 24, 2017”. The icon I41b represents the fact that the patient with patient ID “00000001” has experienced a cardiac failure sign event on event date “Apr. 25, 2017”. The icon I41c represents the fact that the patient with patient ID “00000001” has experienced a cardiac failure sign event on event date “Apr. 26, 2017”. The icon I42 represents the fact that the patient with patient ID “00000002” has experienced a cardiac failure sign event on event date “Apr. 15, 2017”. The icon I43 represents the fact that the patient with patient ID “00000003” has experienced a cardiac failure sign event on event date “Apr. 10, 2017”. The icon I44 represents the fact that the patient with patient ID “00000004” has experienced a cardiac failure sign event on event date “Apr. 14, 2017”.

Note that a linear indicator L1 is displayed in the mapping area R41. The indicator L1 indicates an arbitrary date and time on the time axis included in the mapping area R41. The display position of the indicator L1 can be arbitrarily changed by a user's instruction via the input interface 12, for example, a clicking operation or cursor movement. This equally applies to the indicator displayed in a subsequent mapping area.

The event associated information area R42 includes areas R421, R422, R423, R424, and R425 representing event associated information associated with event information for each patient. The right end of the event associated information area R42 is provided with a scroll bar SC1. A user's instruction (scroll operation) with respect to the scroll bar SC1 can change the display range in the event associated information area R42. In the event associated information area R42 shown in FIG. 4, the areas R423 and R425 are not partially displayed on the display 13.

The area R421 displays cardiac failure sign event associated information about the patient with patient ID “00000001”. The area R421 displays information indicating that the name and sex of the patient with patient ID “00000001” are respectively “Taro Shinzo” and “male”. The area R421 also displays a daily urine output shift in a graphical form in the period from Apr. 10, 2017 to Apr. 27, 2017, which is obtained from the patient with patient ID “00000001”. The area R421 includes a button B41. When the user designates the button B41 via the input interface 12, the processing circuitry 11 displays the details of medical treatment information about the patient with patient ID “00000001”. Accordingly, the user can access the details of medical treatment information about the patient with patient ID “00000001” by designating the button B41. Note that when the user designates an icon mapped to the mapping area, the processing circuitry 11 may display a detailed screen of medical treatment information about the patient associated with the icon. This equally applies to the following case.

The area R421 includes marks M41a, M41b, and M41c that emphasize the time points of the occurrence of cardiac failure sign event in the patient with patient ID “00000001”. The mark M41a represents the occurrence of a cardiac failure sign event on date “Apr. 24, 2017” and corresponds to the icon I41a. The mark M41b represents the occurrence of a cardiac failure sign event on date “Apr. 25, 2017” and corresponds to the icon I41b. The mark M41c represents the occurrence of a cardiac failure sign event on date “Apr. 26, 2017” and corresponds to the icon I41c.

Although the area R421 summarizes and displays a plurality of events in the patient with patient ID “00000001”, this is not exhaustive. For example, the event associated information area R42 may include three areas respectively including the mark M41a, the mark M41b, and the mark 41c.

The area R422 displays cardiac failure sign event associated information about the patient with patient ID “00000002”. The area R422 displays, for example, information indicating that the name and sex of the patient with patient ID “00000002” are respectively “Hanako Shinzo” and “female”. The area R422 also displays a daily urine output shift in a graphical form in the period from Apr. 10, 2017 to Apr. 27, 2017, which is obtained from the patient with patient ID “00000002”. The area R422 includes a button B42. The user can access the details of medical treatment information about the patient with patient ID “00000002” by designating the button B42.

The area R422 includes a mark M42 that emphasizes the time point of the occurrence of a cardiac failure sign event in the patient with patient ID “00000002”. The mark M42 represents the occurrence of a cardiac failure sign event on date “Apr. 15, 2017” and corresponds to the icon I42.

The area R424 displays cardiac failure sign event associated information about the patient with patient ID “00000004”. The area R424 displays, for example, information indicating that the name and sex of the patient with patient ID “00000004” are respectively “Ichiro Shinzo” and “male”. The area R424 also displays a daily urine output shift in a graphical form in the period from Apr. 10, 2017 to Apr. 27, 2017, which is obtained from the patient with patient ID “00000004”. The area R424 includes a button B44. The user can access the details of medical treatment information about the patient with patient ID “00000004” by designating the button B44.

The area R424 includes a mark M44 that emphasizes the time point of the occurrence of a cardiac failure sign event in the patient with patient ID “00000004”. The mark M44 represents the occurrence of a cardiac failure sign event on date “Apr. 14, 2017” and corresponds to the icon I44.

The area R423 displays cardiac failure sign event associated information about the patient with patient ID “00000003”. The area R423 displays, for example, information indicating that the name and sex of the patient with patient ID “00000003” are respectively “Jiro Shinzo” and “male”. The area R423 also displays a daily urine output shift in a graphical form in the period from Apr. 10, 2017 to Apr. 27, 2017, which is obtained from the patient with patient ID “00000003”. The area R423 includes a button B43. The user can access the details of medical treatment information about the patient with patient ID “00000003” by designating the button B43.

The area R423 includes a mark M43 that emphasizes the time point of the occurrence of a cardiac failure sign event in the patient with patient ID “00000003”. The mark M43 represents the occurrence of a cardiac failure sign event on date “Apr. 10, 2017” and corresponds to the icon I43.

The area R425 displays cardiac failure sign event associated information about the patient with patient ID “00000005”. The area R425 displays, for example, information indicating that the name and sex of the patient with patient ID “00000005” are respectively “Saburo Shinzo” and “male”. The area R425 also displays a daily urine output shift in a graphical form in the period from Apr. 10, 2017 to Apr. 27, 2017, which is obtained from the patient with patient ID “00000005”. The area R425 includes a button B45. The user can access the details of medical treatment information about the patient with patient ID “00000005” by designating the button B45.

The area R425 includes a mark M45 that emphasizes the time point of the occurrence of a cardiac failure sign event in the patient with patient ID “00000005”. The mark M45 represents the occurrence of a cardiac failure sign event on date “Apr. 18, 2017” and corresponds to the icon I45.

According to the medical treatment screen displayed in FIG. 4 described above, for example, upon visually checking the mapping data displayed in the mapping area R41, the user can know, at a glance, a specific patient in whom events have concentrated and specific dates on which the events have concentrated. More specifically, referring to FIG. 4, for example, upon visually checking the mapping area R41, the user can recognize, at a glance, that cardiac failure sign event has occurred in the patient with patient ID “00000001” on three consecutive event dates “Apr. 24, 2017”, “Apr. 25, 2017”, and “Apr. 26, 2017”.

Upon visually checking the mapping area R41, for example, the user determines that a medical treatment needs to be preferentially performed on the patient with patient ID “00000001”, and can access the details of medical treatment information concerning the patient. In this case, the user need not check the details of medical treatment information concerning patients other than the patient on which a medical treatment should be preferentially performed. This makes it possible to reduce the frequency of unnecessary screen transition for checks on the details of medical treatment information concerning patients. That is, it is possible to shorten the time for trial and error required to access the details of medical treatment information concerning the patient on which a medical treatment should be preferentially performed.

According to the first embodiment, the processing circuitry 11 of the medical information display apparatus 1 acquires event information for each patient based on predetermined acquisition conditions. Based on the acquired event information, the processing circuitry 11 generates mapping data by mapping event represented by the event information along a common time axis so as to make the events identifiable for each patient. More specifically, the processing circuitry 11 generates mapping data by mapping one or more icons concerning event along a common time axis so as to make the icons identifiable for each patient based on a plurality of pieces of patient identification information and a plurality of pieces of time information.

Upon visually checking the display of the mapping data, the user can know, at a glance, a specific patient in whom events have concentrated and specific dates on which the events have concentrated. The user need not check the details of medical treatment information concerning patients other than the patient on which a medical treatment should be preferentially performed. This makes it possible to shorten the time for trial and error required to access the details of medical treatment information concerning the patient on which a medical treatment should be preferentially performed.

The processing circuitry 11 displays event associated information associated with events together with mapping data. Accordingly, upon visually checking the event associated information, the user can recognize the contents of events without checking the details of medical treatment information concerning the patient.

Note that the mapping data of the event displayed by the processing circuitry 11 of the medical information display apparatus 1 is not limited to data notifying the medical condition of a patient, such as cardiac failure sign event. The mapping data of the event displayed by the processing circuitry 11 may be, for example, data concerning an event in a medical treatment workflow. Events in a medical treatment workflow include a surgery schedule event representing a surgery schedule, an examination result event representing an examination result, and an outpatient consultation event representing an outpatient consultation schedule. The following will describe each of display forms when the processing circuitry 11 displays a surgery schedule event and an image examination event representing an image examination result included in an examination result event.

The first will describe a display form when the processing circuitry 11 displays mapping data about surgery schedule events on the display 13. In this case, the processing circuitry 11 executes processing in the flowchart shown in FIG. 3. Assume that in the following description, the processing circuitry 11 receives the designation of event type “surgery schedule event” via the input interface 12. Assume also that acquisition conditions for the acquisition of event information (surgery schedule event information) representing this surgery schedule event include, for example, “medical treatment information recoded as a surgery schedule”. In addition, this acquisition condition includes temporal elements (for example, a period) and elements representing attributes of a patient (for example, a doctor in charge, medical treatment department, and hospital ward). A temporal element is, for example, “the period from the event date of Apr. 10, 2017 to the event date of Apr. 27, 2017”. An element representing an attribute of a patient is, for example, a patient attended by doctor A. Assume that in this case, the patients attended by doctor A correspond to the patients with patient IDs “00000001”, “00000002”, “00000003”, “00000004”, and “00000005”.

Upon receiving the designation of an acquisition condition, the processing circuitry 11 reads out the designated acquisition condition from the memory 14 (step SA1). The processing circuitry 11 reads out, for example, an acquisition condition for the acquisition of surgery schedule events from the memory 14.

The processing circuitry 11 acquires a plurality of pieces of event information based on the readout acquisition condition (step SA2). The processing circuitry 11 acquires, for example, surgery schedule event information for each patient from the medical treatment information recorded as surgery schedules included in the log information 621 stored in the memory 62 of the DWH 6. In other words, the processing circuitry 11 acquires patient identification information for identifying patients corresponding to the occurrence of surgery schedule events and temporal information representing the times associated with the surgery schedule events.

In this case, the acquired surgery schedule event information includes a record having event type “surgery schedule event”, patient ID “00000001”, event date “Apr. 14, 2017”, event associated information (surgery schedule event associated information) associated with a surgery schedule event, and the like. Surgery schedule event associated information includes, for example, disease name “ventricular septal defect”, age (month old) “3 months”, admission date “2017/4/1”, surgery schedule date “2017/4/14”, operative procedure “BT shunt”, and surgeon “test doctor”. Surgery schedule event information includes a record having event type “surgery schedule event”, patient ID “00000002”, event date “Apr. 14, 2017”, surgery schedule event associated information, and the like. Surgery schedule event associated information includes, for example, disease name “Fallot's tetralogy”, age (month old) “3 years”, admission date “2017/4/8”, surgery schedule date “2017/4/14”, operative procedure “BT shunt”, and surgeon “test doctor”. Surgery schedule event information includes a record having event type “surgery schedule event”, patient ID “00000003”, event date “Apr. 21, 2017”, surgery schedule event associated information, and the like. Surgery schedule event associated information includes, for example, disease name “ventricular septal defect”, age (month old) “4 years”, admission date “2017/4/5”, surgery schedule date “2017/4/21”, operative procedure “BT shunt”, and surgeon “test doctor”. Surgery schedule event information includes a record having event type “surgery schedule event”, patient ID “00000004”, event date “Apr. 21, 2017”, surgery schedule event associated information, and the like. Surgery schedule event associated information includes, for example, disease name “hypoplastic left heart syndrome”, age (month old) “5 years”, admission date “2017/4/4”, surgery schedule date “2017/4/21”, operative procedure “Fontan surgery”, and surgeon “test doctor”. Surgery schedule event information includes a record having event type “surgery schedule event”, patient ID “00000005”, event date “Apr. 26, 2017”, surgery schedule event associated information, and the like. Surgery schedule event associated information includes, for example, disease name “ventricular septal defect”, age (month old) “2 years”, admission date “2017/4/6”, surgery schedule date “2017/4/26”, operative procedure “BT shunt”, and surgeon “test doctor”.

The processing circuitry 11 maps the plurality of pieces of acquired event information about a plurality of patients (step SA3). More specifically, the processing circuitry 11 generates, for example, mapping data by mapping one or more icons concerning surgery schedule events along a common time axis so as to make the icons identifiable for each patient based on a plurality of pieces of acquired patient identification information and a plurality of pieces of acquired time information.

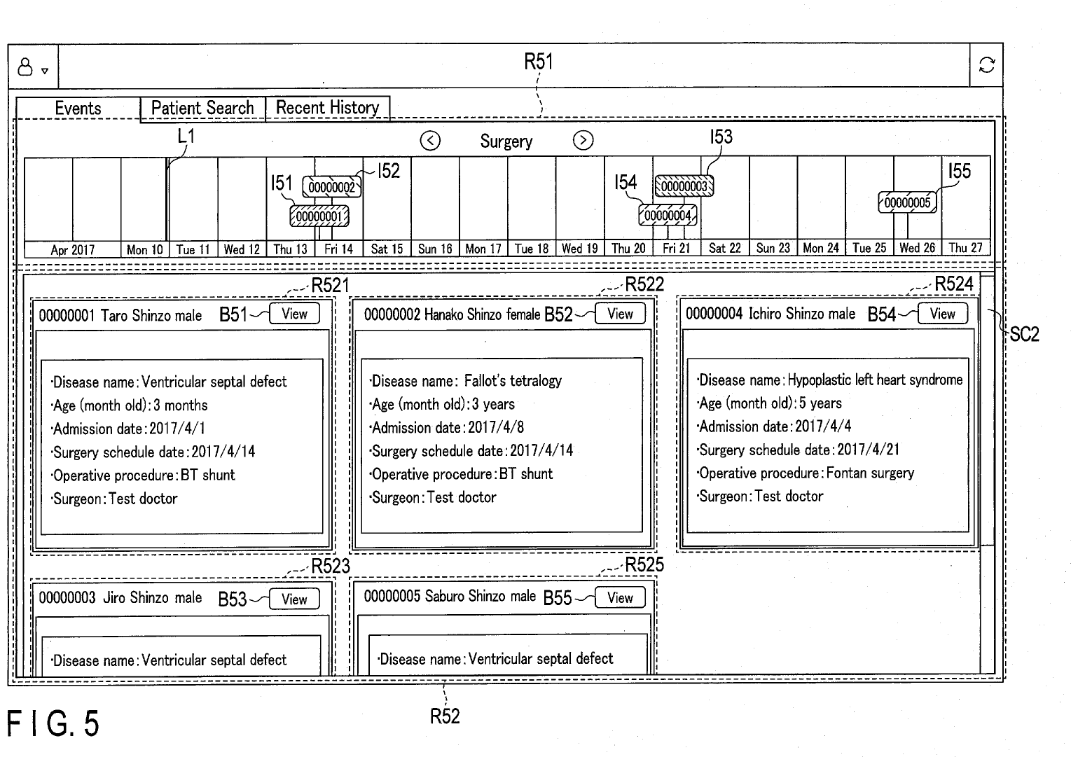

The processing circuitry 11 displays the event associated information concerning events for each patient together with the mapping data (step SA4). FIG. 5 is a view showing an example of a medical treatment screen concerning the surgery schedule events displayed on the display 13 shown in FIG. 2. The medical treatment screen displayed in FIG. 5 includes a mapping area R51 having mapping data and an event associated information area R52 having at least one pieces of event associated information.

The mapping area R51 includes icons I51, I52, I53, I54, and I55 respectively representing patients. As shown in FIG. 5, the respective icons included in the mapping area R51 are displayed in different colors for each patient. Note that, the respective icons may display with patient name.

The icon I51 represents the fact that a surgery is scheduled for the patient with patient ID “00000001” on event date “Apr. 14, 2017”. The icon I52 represents the fact that a surgery is scheduled for the patient with patient ID “00000002” on event date “Apr. 14, 2017”. The icon I53 represents the fact that a surgery is scheduled for the patient with patient ID “00000003” on event date “Apr. 21, 2017”. The icon I54 represents the fact that a surgery is scheduled for the patient with patient ID “00000004 on event date “Apr. 21, 2017”. The icon I55 represents the fact that a surgery is scheduled for the patient with patient ID “00000005” on event date “Apr. 26, 2017”.

The event associated information area R52 includes areas R521, R522, R523, R524, and R525 representing event associated information associated with event information for each patient. The right end of the event associated information area R52 is provided with a scroll bar SC2. A user's instruction with respect to the scroll bar SC2 can change the display range in the event associated information area R52. In the event associated information area R52 shown in FIG. 5, the areas R523 and R525 are not partially displayed on the display 13.

The area R521 displays surgery schedule event associated information about the patient with patient ID “00000001”. The area 8521 displays information indicating that the name and sex of the patient with patient ID “00000001” are respectively “Taro Shinzo” and “male”. The area R521 also displays, concerning the patient with patient ID “00000001”, disease name “ventricular septal defect”, age (month old) “3 months”, admission date “2017/4/1”, surgery schedule date “2017/4/14”, operative procedure “BT shunt”, and surgeon “test doctor”. The area R521 includes a button B51. When the user designates the button B51 via the input interface 12, the processing circuitry 11 displays the details of medical treatment information about the patient with patient ID “00000001”. Accordingly, the user can access the details of medical treatment information about the patient with patient ID “00000001” by designating the button B51.

The area R522 displays surgery schedule event associated information about the patient with patient ID “00000002”. The area R522 displays information indicating that the name and sex of the patient with patient ID “00000002” are respectively “Hanako Shinzo” and “female”. The area R522 also displays disease name “Fallot's tetralogy”, age (month old) “3 years”, admission date “2017/4/8”, surgery schedule date “2017/4/14”, operative procedure “BT shunt”, and surgeon “test doctor”. The area R522 includes a button B52. The user can access the details of medical treatment information about the patient with patient ID “00000002” by designating the button B52.

The area R524 displays surgery schedule event associated information about the patient with patient ID “00000004”. The area R524 displays information indicating that the name and sex of the patient with patient ID “00000004” are respectively “Ichiro Shinzo” and “male”. The area R524 also displays, concerning the patient with patient ID “00000004”, disease name “hypoplastic left heart syndrome”, age (month old) “5 years”, admission date “2017/4/4”, surgery schedule date “2017/4/21”, operative procedure “Fontan surgery”, and surgeon “test doctor”. The area R524 includes a button B54. The user can access the details of medical treatment information about the patient with patient ID “00000004” by designating the button B54.

The area R523 displays surgery schedule event associated information about the patient with patient ID “00000003”. The area R523 displays information indicating that the name and sex of the patient with patient ID “00000003” are respectively “Jiro Shinzo” and “male”. The area R523 also displays disease name “ventricular septal defect” concerning the patient with patient ID “00000003”. Note that part of the event associated information is not displayed in the area R523. In addition, the area R523 includes a button B53. The user can access the details of medical treatment information about the patient with patient ID “00000003” by designating the button B53.

The area R525 displays surgery schedule event associated information about the patient with patient ID “00000005”. The area R525 displays information indicating that the name and sex of the patient with patient ID “00000005” are respectively “Saburo Shinzo” and “male”. The area R525 also displays disease name “ventricular septal defect” concerning the patient with patient ID “00000005”. Note that part of the event associated information is not displayed in the area R525. In addition, the area R525 includes a button B55. The user can access the details of medical treatment information about the patient with patient ID “00000005” by designating the button B55.

The next will describe a display form when the processing circuitry 11 displays mapping data concerning image examination events on the display 13. In this case, the processing circuitry 11 executes processing in the flowchart shown in FIG. 3. Assume that in the following description, the processing circuitry 11 receives the designation of event type “image examination event” via the input interface 12. Assume also that acquisition conditions for the acquisition of event information (image examination event information) representing this image examination event include, for example, “medical treatment information representing an image examination result”. In addition, this acquisition condition includes temporal elements (for example, a period) and elements representing attributes of a patient (for example, a doctor in charge, medical treatment department, and hospital ward). A temporal element is, for example, “the period from the event date Apr. 10, 2017 to the event date Apr. 27, 2017”. An element representing an attribute of a patient is, for example, a patient attended by doctor A. Assume that in this case, the patients attended by doctor A correspond to the patients with patient IDs “00000001”, “00000002”, “00000003”, “00000004”, and “00000005”.

Upon receiving the designation of an acquisition condition, the processing circuitry 11 reads out the designated acquisition condition from the memory 14 (step SA1). The processing circuitry 11 reads out, for example, an acquisition condition for the acquisition of image examination events from the memory 14.

The processing circuitry 11 acquires a plurality of pieces of event information based on the readout acquisition condition (step SA2). The processing circuitry 11 acquires, for example, event information representing image examination events for each patient from the medical treatment information representing image examination results included in the log information 621 stored in the memory 62 of the DWH 6 based on the readout acquisition condition for the acquisition of image examination events. In other words, the processing circuitry 11 acquires patient identification information for identifying patients corresponding to the occurrence of image examination events and temporal information representing the times associated with the image examination events.

In this case, the acquired image examination event information includes a record having event type “image examination event”, patient ID “00000001”, event date “Apr. 23, 2017”, event associated information (image examination event associated information) associated with the image examination event, and the like. The image examination event associated information includes, for example, medical image data acquired concerning the patient with patient ID “00000001”. The image examination event information includes a record having event type “image examination event”, patient ID “00000001”, event date “Apr. 22, 2017”, and image examination event associated information. The image examination event associated information is, for example, medical image data acquired concerning the patient with patient ID “00000002”. The image examination event information also includes a record having event type “image examination event”, patient ID “00000003”, event date “Apr. 18, 2017”, and image examination event associated information. The image examination event associated information is, for example, medical image data acquired concerning the patient with patient ID “00000003”. The image examination event information also includes a record having event type “image examination event”, patient ID “00000004”, event date “Apr. 19, 2017”, image examination event associated information, and the like. The image examination event associated information is, for example, medical image data acquired concerning the patient with patient ID “00000004”. The image examination event information also includes a record having event type “image examination event”, patient ID “00000005”, event date “Apr. 13, 2017”, image examination event associated information, and the like. The image examination event associated information is, for example, medical image data acquired concerning the patient with patient ID “00000005”.

The processing circuitry 11 maps the plurality of pieces of acquired event information about a plurality of patients (step SA3). More specifically, the processing circuitry 11 generates, for example, mapping data by mapping one or more icons concerning surgery schedule events along a common time axis so as to make the icons identifiable for each patient based on a plurality of pieces of acquired patient identification information and a plurality of pieces of acquired time information.

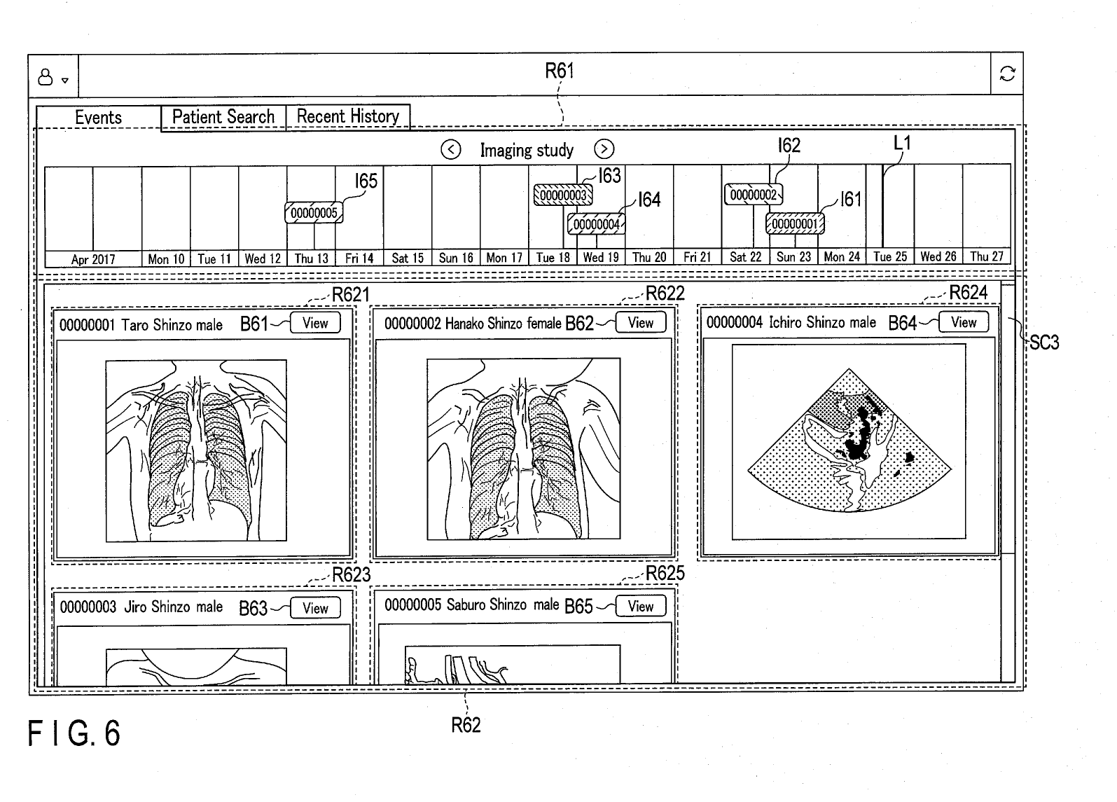

The processing circuitry 11 displays the event associated information concerning events for each patient together with the mapping data (step SA4). FIG. 6 is a view showing an example of a medical treatment screen concerning the image examination events displayed on the display shown in FIG. 2. The medical treatment screen displayed in FIG. 6 includes a mapping area R61 having mapping data and an event associated information area R62 having at least one piece of event associated information.

The mapping area R61 includes icons I61, I62, I63, I64, and I65 respectively representing patients. As shown in FIG. 6, the respective icons included in the mapping area R61 are displayed in different colors for each patient. Note that, the respective icons may display with patient name.

The icon I61 represents the fact that an image examination, that is, the acquisition of a medical image, has been performed concerning the patient with patient ID “00000001” on event date “Apr. 23, 2017”. The icon I62 represents the fact that an image examination has been performed with respect to the patient with patient ID “00000002” on event date “Apr. 22, 2017”. The icon I63 represents the fact that an image examination has been performed with respect to the patient with patient ID “00000003” on event date “Apr. 18, 2017”. The icon I64 represents the fact that an image examination has been performed with respect to the patient with patient ID “00000004” on event date “Apr. 19, 2017”. The icon I65 represents the fact that an image examination has been performed with respect to the patient with patient ID “00000005” on event date “Apr. 13, 2017”.

The event associated information area R62 includes areas R621, R622, R623, R624, and R625 representing event associated information associated with event information for each patient. The right end of the event associated information area R62 is provided with a scroll bar SC3. A user's instruction with respect to the scroll bar SC3 can change the display range in the event associated information area R62. In the event associated information area R62 shown in FIG. 6, the areas R623 and R625 are not partially displayed on the display 13.

The area R621 displays image examination event associated information about the patient with patient ID “00000001”. The area R621 displays information indicating that the name and sex of the patient with patient. ID “00000001” are respectively “Taro Shinzo” and “male”. The area R621 displays the medical image based on the medical image data acquired concerning the patient with patient ID “00000001” on event date “Apr. 23, 2017”. The area R621 includes a button B61. When the user designates the button B61 via the input interface 12, the processing circuitry 11 displays the details of medical treatment information about the patient with patient ID “00000001”. Accordingly, the user can access the details of medical treatment information about the patient with patient ID “00000001” by designating the button B61.

The area R622 displays image examination event associated information about the patient with patient ID “00000002”. The area R622 displays information indicating that the name and sex of the patient with patient ID “00000002” are respectively “Hanako Shinzo” and “female”. The area R622 displays the medical image based on the medical image data acquired concerning the patient with patient ID “00000002” on event date “Apr. 22, 2017”. The area R622 includes a button B62. The user can access the details of medical treatment information about the patient with patient ID “00000002” by designating the button B62.

The area R624 displays image examination event associated information about the patient with patient ID “00000004”. The area R624 displays information indicating that the name and sex of the patient with patient ID “00000004” are respectively “Ichiro Shinzo” and “male”. The area R624 displays the medical image based on the medical image data acquired concerning the patient with patient ID “00000004” on event date “Apr. 19, 2017”. The area R624 includes a button B64. The user can access the details of medical treatment information about the patient with patient ID “00000004” by designating the button B64.

The area R623 displays image examination event associated information about the patient with patient ID “00000003”. The area R623 displays information indicating that the name and sex of the patient with patient ID “00000003” are respectively “Jiro Shinzo” and “male”. The area R623 displays the medical image based on the medical image data acquired concerning the patient with patient ID “00000003” on event date “Apr. 18, 2017”. Note that part of the medical image is not displayed in the area R623. The area R623 includes a button B63. The user can access the details of medical treatment information about the patient with patient ID “00000003” by designating the button B63.

The area R625 displays image examination event associated information about the patient with patient ID “00000005”. The area R625 displays information indicating that the name and sex of the patient with patient ID “00000005” are respectively “Saburo Shinzo” and “male”. The area R625 displays the medical image based on the medical image data acquired concerning the patient with patient ID “00000005” on event date “Apr. 13, 2017”. Note that part of the medical image is not displayed in the area R625. The area R625 includes a button B65. The user can access the details of medical treatment information about the patient with patient ID “00000005” by designating the button B65.

(First Modification)

According to the above embodiment, the processing circuitry 11 generates summary screen data concerning one type of event. However, this is not exhaustive. The processing circuitry 11 may generate summary screen data by summarizing event information concerning a plurality of patients and a plurality of types of events. Assume that in the first modification, the processing circuitry 11 receives the designation of an acquisition condition concerning a plurality of types of events via the input interface 12. Assume that in this case, the processing circuitry 11 displays, for example, a plurality of areas that enables the selection of character strings representing acquisition condition candidates in a pull-down form on the display 13, and receives the designation of an acquisition condition concerning a plurality of types of events.

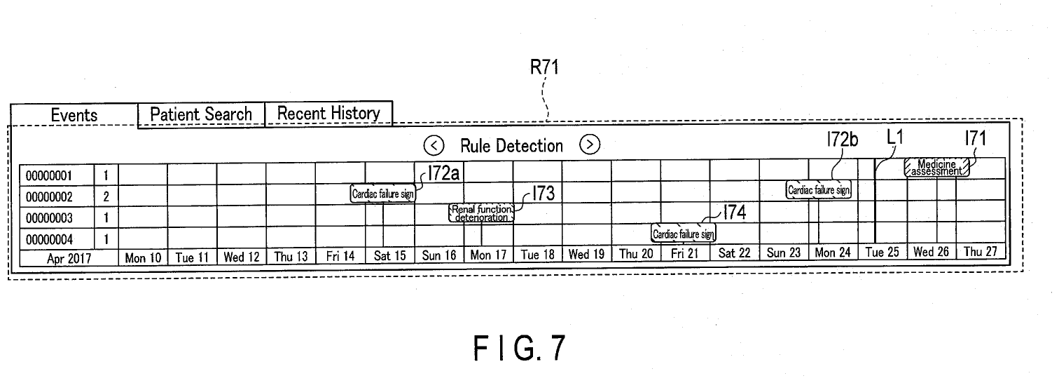

FIG. 7 is a view showing a display example of mapping data according to the first modification. Assume that, referring to FIG. 7, the processing circuitry 11 has received, for example, the designation of event types “cardiac failure sign event”, “renal function deterioration event”, and “medicine assessment event”. The mapping data shown in FIG. 7 is displayed on, for example, the display 13 shown in FIG. 2.

Referring to FIG. 7, icons including character strings representing event types are mapped in time series. More specifically, in a mapping area R71 shown in FIG. 7, icons I71, I72a, I72b, I73, and I74 respectively representing event types are mapped in time series, with the ordinate representing the patient IDs and the abscissa representing the time (date). The display period of events shown in FIG. 7 is the period from Apr. 10, 2017 to Apr. 27, 2017.

More specifically, the icon I71 including “medicine assessment” representing a medicine assessment event is mapped at a position corresponding to patient ID “00000001” and date “Apr. 26, 2017”. The icon I72a including “cardiac failure sign” representing a cardiac failure sign event is mapped at a position corresponding to patient ID “00000002” and date “Apr. 15, 2017”. The icon I72b including “cardiac failure sign” representing a cardiac failure sign event is mapped at a position corresponding to patient ID “00000002” and date “Apr. 24, 2017”. The icon I73 including “renal function deterioration” representing a renal function deterioration event is mapped at a position corresponding to patient ID “00000003” and date “Apr. 17, 2017”. The icon I74 including “cardiac failure sign” representing a cardiac failure sign event is mapped at a position corresponding to patient ID “00000004” and date “Apr. 17, 2017”.

The numbers “1”, “2”, “1”, and “1” displayed beside the respective patient IDs shown in FIG. 7 respectively represent the numbers of events displayed in the mapping area R71. Note that the numbers displayed beside the respective patient IDs may represent the numbers of events that are not displayed in the mapping area R71 or the sums of the numbers of events displayed and the numbers of events that are not displayed, with respect to the respective patient IDs, when, for example, acquired event information exists outside the display period.

As shown in FIG. 7, for example, the respective icons included in the mapping area R71 are displayed in different colors for each event type.

According to the display form shown in FIG. 7 described above, upon visually checking the mapping data shown in FIG. 7, for example, the user can obtain more information without a deterioration in visibility, because a plurality of types of events are displayed in time series for each patient.

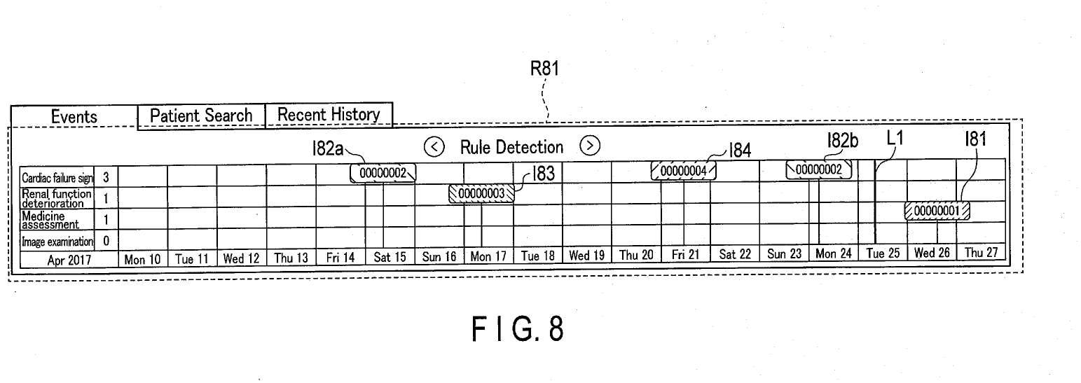

FIG. 8 shows another display example of mapping data according to the first modification. Assume that, referring to FIG. 8, the processing circuitry 11 has received, for example, the designation of event types “cardiac failure sign event”, “renal function deterioration event”, “medicine assessment event”, and “image examination event”. The mapping data shown in FIG. 8 is displayed on, for example, the display 13 shown in FIG. 2.

Referring to FIG. 8, icons including patient IDs representing patients are mapped in time series for each event type. More specifically, in a mapping area R81 shown in FIG. 8, icons I81, I82a, I82b, I83, and I84 respectively representing patient IDs specifying the respective patients are mapped in time series, with the ordinate representing character strings representing the respective event types and the abscissa representing the time (date). The display period of events shown in FIG. 8 is the period from Apr. 10, 2017 to Apr. 27, 2017. Note that, the respective icons may display with patient name.

More specifically, the icon I81 including patient ID “00000001” is mapped at a position corresponding to character string “medicine assessment” representing an medicine assessment event and date “Apr. 26, 2017”. The icon I82a including patient ID “00000002” is mapped at a position corresponding to character string “cardiac failure sign” representing a cardiac failure sign event and date “Apr. 15, 2017”. The icon I82b including patient ID “00000002” is mapped at a position corresponding to character string “cardiac failure sign” representing a cardiac failure sign event and date “Apr. 24, 2017”. The icon I83 including patient ID “00000003” is mapped at a position corresponding to character string “renal function deterioration” representing a renal function deterioration event and date “Apr. 17, 2017”. The icon I84 including patient ID “00000004” is mapped at a position corresponding to character string “cardiac failure sign” representing a cardiac failure sign event and date “Apr. 21, 2017”.

The numbers “3”, “1”, “1”, and “0” displayed beside the character strings representing the respective event types shown in FIG. 8 respectively represent the total numbers of patients displayed in the mapping area R81 for the respective event types. Note that the numbers displayed beside the character strings representing the respective event types represent, for example, the numbers of patients who are not displayed in the mapping area R81 or the sums of the numbers of patients who are displayed and the numbers of patients who are not displayed, for the respective event types, when acquired event information exists outside the display period.

As shown in FIG. 8, for example, the respective icons included in the mapping area R81 are displayed in different colors for each event type.

According to the display form shown in FIG. 8 described above, upon visually checking the mapping data shown in FIG. 8, for example, the user can obtain more information without a deterioration in visibility, because a plurality of patients attended by the user are displayed for each event type in time series.

Referring to FIG. 8, data are collectively displayed in areas including one time axis. However, this is not exhaustive. The following will describe a case in which mapping data are displayed, with areas including one time axis being assigned to events associated with the respective event types.

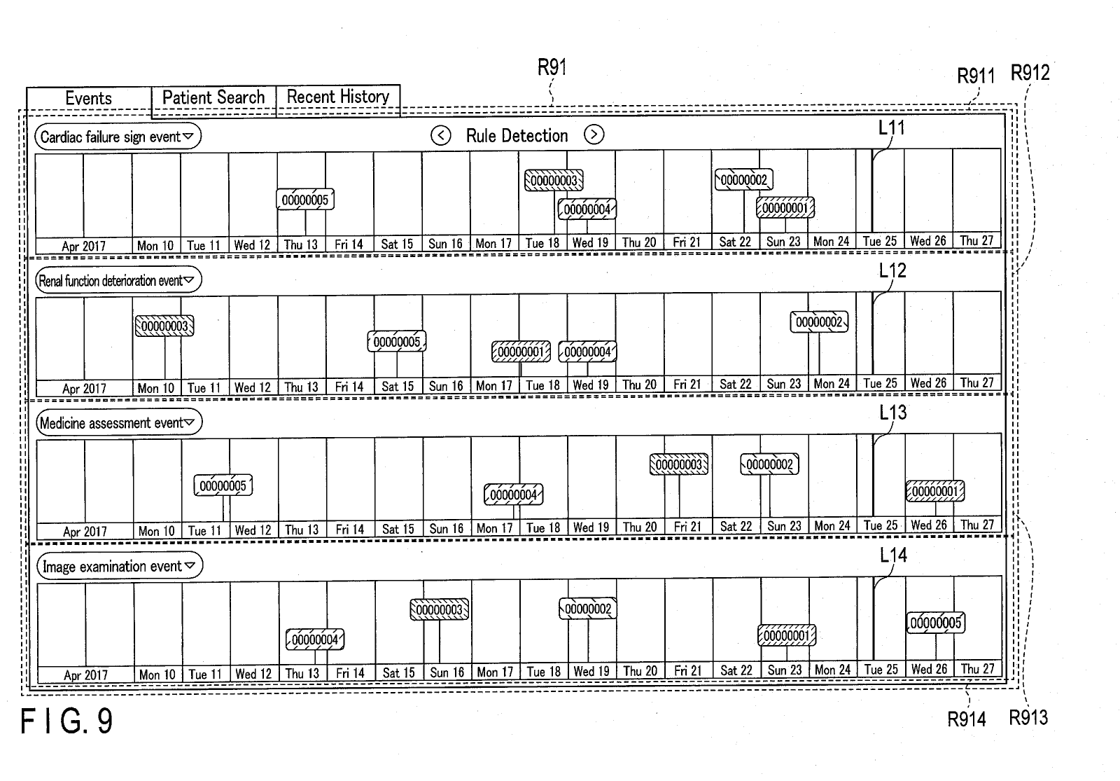

FIG. 9 is a view showing another display example of mapping data according to the first modification. Assume that referring to FIG. 9, the processing circuitry 11 has received, for example, the designation of character strings “cardiac failure sign event”, “renal function deterioration event”, “medicine assessment event”, and “image examination event”.

Referring to FIG. 9, a mapping area R91 includes areas R911, R912, R913, and R914 including one time axis for each event type. The area R911 displays mapping data by mapping cardiac failure sign event along a common time axis so as to make the events identifiable for each patient. The area R912 displays mapping data by mapping renal function deterioration events along a common time axis so as to make the events identifiable for each patient. The area R913 displays mapping data by mapping medicine assessment events along a common time axis so as to make events identifiable for each patient. The area R914 displays mapping data by mapping image examination events along a common time axis so as to make the events identifiable for each patient.

According to the display form shown in FIG. 9 described above, for example, a large display area per event type improves visibility as compared the mapping data shown in FIG. 8.

(Second Modification)

In the above embodiment, the processing circuitry 11 may display, for example, each event included in the mapping data shown in FIG. 4 in consideration of the browse history of the user. For example, the processing circuitry 11 displays events so as to allow the user to discriminate whether he/she has browsed the events. In this case, that the user browses events is equivalent to that the user accesses the details of medical treatment information about a patient which is linked to one event included in mapping data to open a medical treatment screen displaying the details.

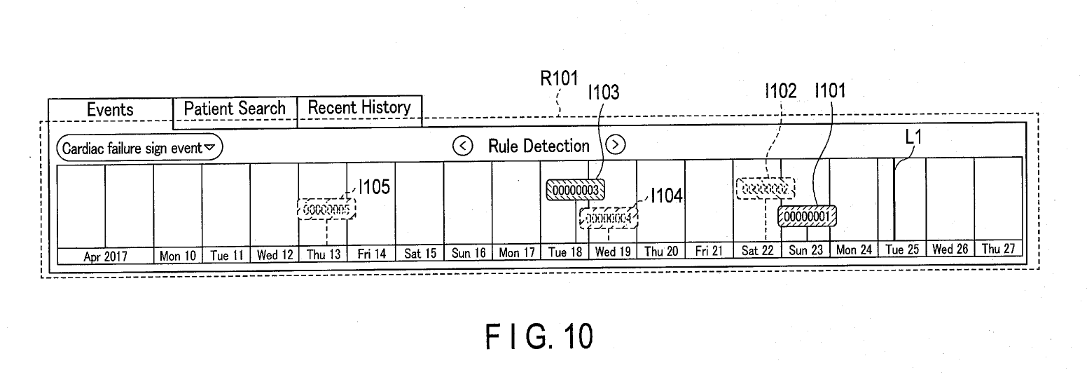

FIG. 10 is a view showing a display example of mapping data according to the second modification. A mapping area R101 shown in FIG. 10 displays the mapping data obtained by mapping cardiac failure sign event concerning a plurality of patients along a common time axis so as to make the events identifiable for each patient.

The mapping area R101 includes icons I101, I102, I103, I104, and I105 representing the respective patients. In this case, the user has already browsed the details of medical treatment information of the patients linked to the respective events represented by the icons I102, I104, and I105 of the icons I101, I102, I103, I104, and I105 shown in FIG. 10. Accordingly, the processing circuitry 11 displays the icons I102, I104, and I105 upon changing the display form. More specifically, the processing circuitry 11 displays the icons I102, I104, and I105 upon changing the frame borders of the respective icons and the patient IDs included in the icons from solid lines to dotted lines.

The display form in FIG. 10 described above allows the user, who has viewed the icons included in the mapping data shown in FIG. 10, to easily grasp whether the user has browsed the events represented by the viewed icons.

According to the description made with reference to FIG. 10, the display form of icons representing the events browsed by the user is changed. However, this is not exhaustive. For example, the processing circuitry 11 may display the display periods indicated in mapping data so as to allow the user to discriminate periods during which the user has browsed from periods during which the user has not browsed. The “period during which the user has browsed” is a period including the event browsed by the user. In addition, the “period during which the user has browsed” may be defined by making the user directly designate a period during which the user thinks that he/she has browsed, with respect to displayed mapping data via the input interface 12.

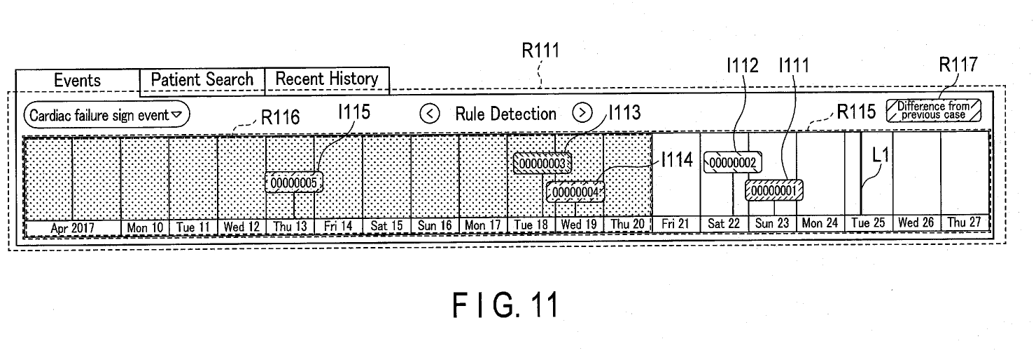

FIG. 11 is a view showing another display example of mapping data according to the second modification. A mapping area R111 shown in FIG. 11 represents the mapping data obtained by mapping cardiac failure sign event concerning a plurality of patients along a common time axis so as to make the events identifiable for each patient.

The mapping area Rill includes icons I111, I112, I113, I114, and I115 representing the respective patients. In this case, the user has already browsed “the period from Apr. 10, 2017 to Apr. 20, 2017”, represented by an area R116, of “the period from Apr. 10, 2017 to Apr. 27, 2017” represented by an area R115 shown in FIG. 11. Accordingly, the processing circuitry 11 displays the area R116 upon changing the display form. More specifically, the processing circuitry 11 displays the area R116 upon superimposing a hatching image. In this case, the hatching image is also superimposed on icons I123, I124, and I125 included in the area R116.

A notification area R117 included in the mapping area R111 shown in FIG. 11 has a function of notifying that the details of medical treatment information about a patient is updated, by designating the area, when the details of the medical treatment information about the patient which has been browsed once is updated. The notification area R117 shown in FIG. 11 indicates a state before designation in a color with low intensity.

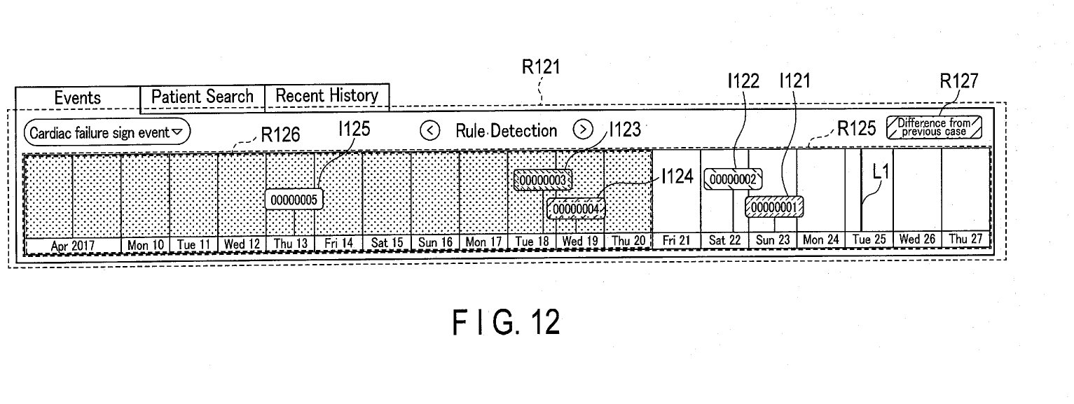

The next will describe a case in which the notification area R117 shown in FIG. 11 is designated. FIG. 12 is a view showing another display example of displayed mapping data according to the second modification. A mapping area R121 shown in FIG. 12 indicates mapping data after the notification area R117 shown in FIG. 11 is designated. In this case, a notification area R127 shown in FIG. 12 indicates a state after designation in, for example, a color with high intensity.

The mapping area R121 includes icons I121, I122, I123, I124, and I125 representing the respective patients. In this case, the details of medical treatment information about the patient corresponding to the event represented by the icon I125 of the icons I121, I122, I123, I124, and I125 shown in FIG. 12 are updated. Accordingly, the processing circuitry 11 cancels the superimposition of only part of the hatching image which corresponds to the area occupied by the icon I125. This makes it possible to notify that the details of the medical treatment information about the patient corresponding to the event represented by the icon I125 has been updated. Note that the processing circuitry 11 may newly display an event that has newly occurred in the period during which the user has browsed.

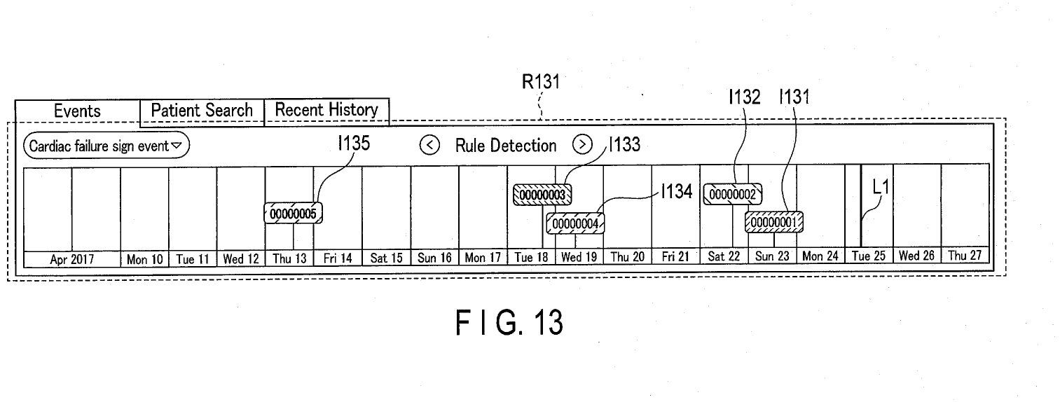

When mapping data is displayed, the processing circuitry 11 may highlight an icon representing an event, of the events included in the mapping data, concerning which, for example, the number of times the details of medical treatment information of the corresponding patient have not been browsed has exceeded a predetermined threshold. FIG. 13 is a view showing another display example of mapping data according to the second modification. A mapping area R131 shown in FIG. 13 indicates mapping data obtained by mapping cardiac failure sign event concerning a plurality of patients along a common time axis so as to make the events identifiable for each patient.

The mapping area R131 includes icons I131, I132, I133, I134, and I135 representing the respective patients. In this case, the numbers of times the user has not browsed the details of medical treatment information of the patients linked to the respective events represented by the icons I133 and I135, of the icons I131, I132, I133, I134, and I135 shown in FIG. 13, do not exceed a predetermined threshold. Accordingly, the processing circuitry 11 highlights the patient IDs included in the icons I133 and I135 in, for example, bold font or by blinking. This makes it possible to notify the user of events overlooked many times.

(Third Modification)

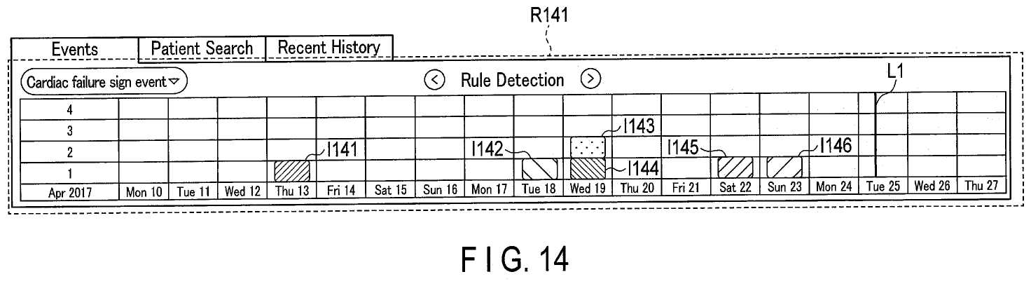

In the above embodiment, the processing circuitry 11 can further improve visibility by devising the display form. For example, the processing circuitry 11 summarizes and displays event information in a histogram or line chart form. The following will describe a case in which event information is summarized and displayed in a histogram form. FIG. 14 is a view showing a display example of mapping data according to the third modification. A mapping area R141 shown in FIG. 14 indicates mapping data obtained by mapping cardiac failure sign event concerning a plurality of patients along a common time axis so as to make the events identifiable for each patient. The mapping area R141 shown in FIG. 14 displays the numbers of events for each date so as to make the numbers recognizable.

More specifically, in the mapping area R141 shown in FIG. 14, icons I141, I142, I143, I144, I145, and I146 representing the patients are mapped in time series, with the ordinate representing the number of events and the abscissa representing the time (date). The display period shown in FIG. 14 is the period from Apr. 10, 2017 to Apr. 27, 2017. Note that referring to FIG. 14, the respective icons included in the mapping area R141 are attached with different colors for each patient so as to make each patient identifiable. The numbers indicated on the left side of the mapping area R141 correspond to the numbers of icons stacked in areas corresponding to the respective dates, that is, the numbers of events.

The display form in FIG. 14 described above allows the user who has visually checked mapping data to easily grasp, for example, the occurrence frequencies of events on the respective dates.

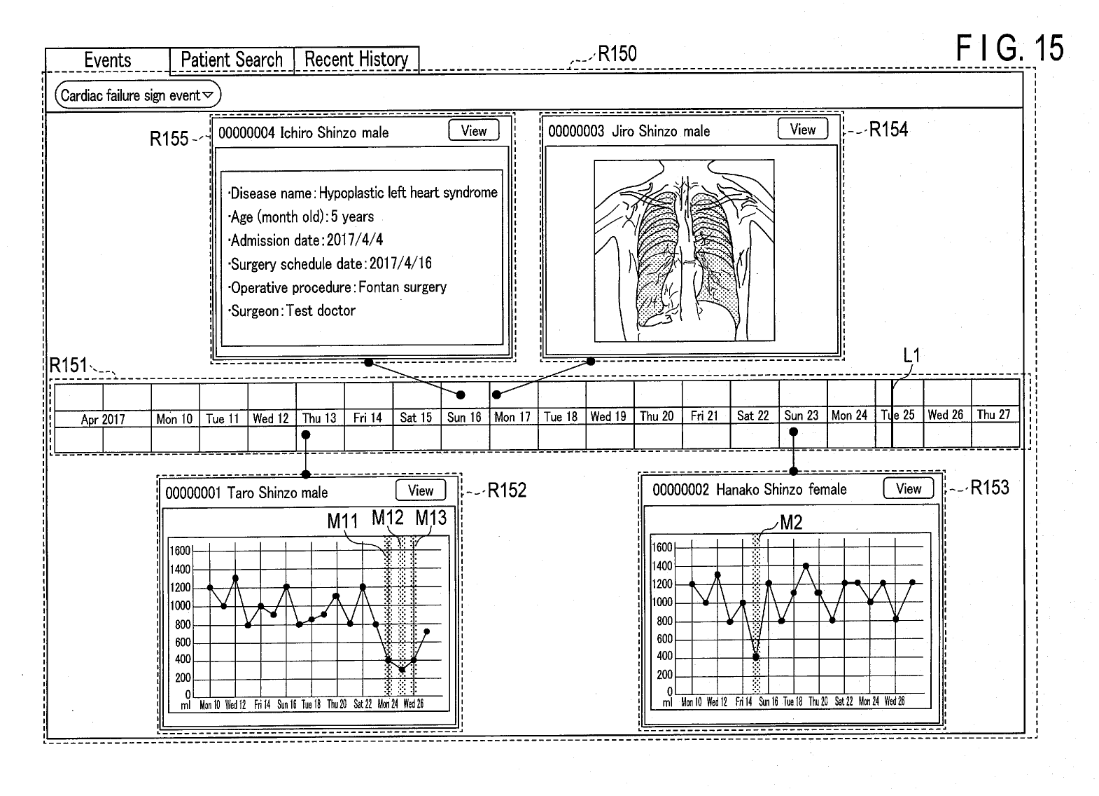

The processing circuitry 11 may also display event associated information upon directly mapping the information along a time axis included in mapping data. FIG. 15 is a view showing another display example of mapping data according to the third modification. A mapping area R150 shown in FIG. 15 indicates mapping data obtained by mapping cardiac failure sign event concerning a plurality of patients along a common time axis so as to make the events identifiable for each patient. The mapping area R150 includes a time axis display area R151 representing a time axis and areas R152, R153, R154, and R155 representing event associated information associated with event information for each patient. As shown in FIG. 15, the areas R152, R153, R154, and R155 are directly mapped in the time axis display area R151. More specifically, the time axis display area R151 is connected to the areas R152, R153, R154, and R155 with line segments.

According to the display form in FIG. 15 described above, because the occurrence timings of events and event associated information are displayed while they are visually linked to each other, the visibility improves.

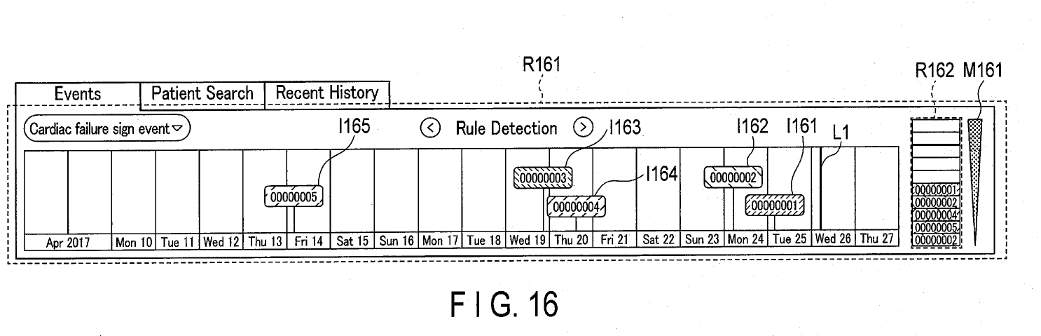

The processing circuitry 11 may further display the priority order of events included in mapping data. FIG. 16 is a view showing another display example of mapping data according to the third modification. A mapping area R161 shown in FIG. 16 indicates mapping data obtained by mapping cardiac failure sign event concerning a plurality of patients along a common time axis so as to make the events identifiable for each patient.

The mapping area R161 includes icons I161, I162, I163, I164, and I165 representing the respective patients. As shown in FIG. 16, the respective icons included in the mapping area R161 are displayed in different colors for each patient. Note that, the respective icons may display with patient name.

The mapping area R161 includes an area R162 representing a queue of events and a mark M161 representing the priority order of processes. The area R162 represents an event queue having stacked icons representing patients. The mark M161 represents higher priority levels with an increase in the thickness of the mark in the vertical direction. That is, a patient corresponding to an icon located at a higher position in the event queue shown in the area R162 has higher priority in terms of checking the details of medical treatment information.

According to the display form in FIG. 16 described above, upon visually checking mapping data, the user can easily grasp priority levels in checking medical treatment information about patients among the patients.

(Fourth Modification)

In the above embodiment, the processing circuitry 11 may change the display form of mapping data displayed in accordance with the appearance pattern of events associated with the same patient. Assume that in the following description, the mapping data displayed on the display 13 includes an area that allows the selection of a summarizing method with a pull-down form.

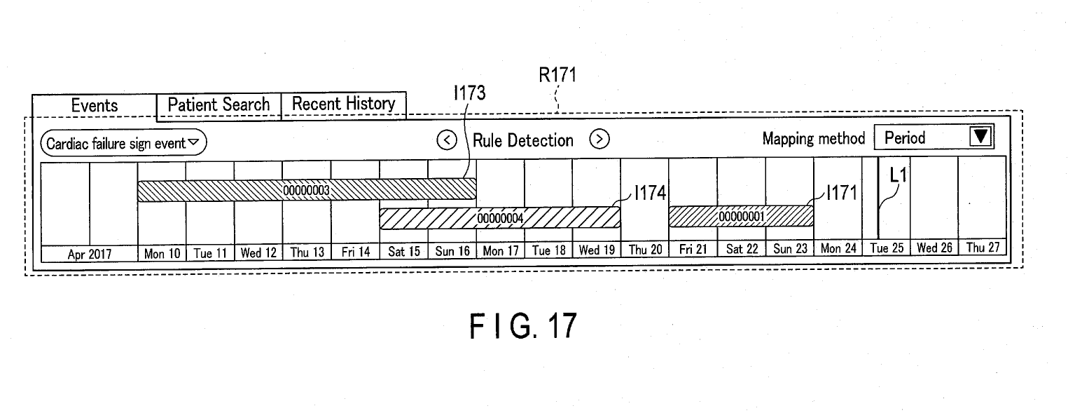

The first will describe a case in which the processing circuitry 11 displays a period including an event group that has occurred in a specific period. FIG. 17 is a view showing a display example of mapping data according to the fourth modification. A mapping area R171 shown in FIG. 17 indicates mapping data obtained by mapping cardiac failure sign event concerning a plurality of patients along a common time axis so as to make the events identifiable for each patient. Referring to FIG. 17, “period” is selected as a mapping method associated with mapping data.

Referring to FIG. 17, the mapping area R171 includes icons I171, I173, and I174 representing the respective patients. The icon I171 represents, in a strip form, the fact that a plurality of cardiac failure sign event have occurred, concerning the patient with patient ID “00000001”, during, for example, “the period from Apr. 21, 2017 to Apr. 23, 2017”. The icon I173 represents, in a strip form, the fact that a plurality of cardiac failure sign event has occurred, concerning the patient with patient ID “00000003”, during, for example, “the period from Apr. 10, 2017 to Apr. 16, 2017”. The icon I174 represents, in a strip form, the fact that a plurality of cardiac failure sign event has occurred, concerning the patient with patient ID “00000004”, during, for example, “the period from Apr. 15, 2017 to Apr. 19, 2017”.

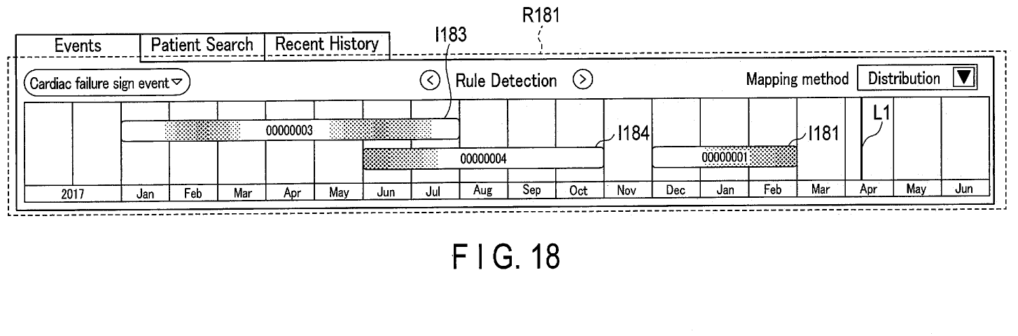

The next will describe a case in which when displaying a period including an event group that has occurred in a specific period, the processing circuitry 11 changes the display form of mapping data in consideration of the occurrence frequency of events. FIG. 18 is a view showing another display example of mapping data according to the fourth modification. A mapping area R181 shown in FIG. 18 indicates mapping data obtained by mapping cardiac failure sign event concerning a plurality of patients along a common time axis so as to make the events identifiable for each patient. Referring to FIG. 18, “distribution” is selected as a mapping method associated with mapping data.

Referring to FIG. 18, the mapping area R181 includes icons I181, I183, and I184 representing the respective patients. The icon I181 represents, in a strip form, the fact that a plurality of cardiac failure sign event has occurred, concerning the patient with patient ID “00000001”, during, for example, “the period from December 2017 to February 2018”. The icon I181 displays the distribution of cardiac failure sign event in grayscale in accordance with the occurrence frequency of the events. The icon I183 represents, in a strip form, the fact that a plurality of cardiac failure sign event has occurred, concerning the patient with patient ID “00000003”, during, for example, “the period from January 2017 to July 2017”. The icon I183 displays the distribution of cardiac failure sign event in grayscale in accordance with the occurrence frequency of the events. The icon I184 represents, in a strip form, the fact that a plurality of cardiac failure sign event has occurred, concerning the patient with patient ID “00000004”, during, for example, “the period from June 2017 to October 2017”. The icon I184 displays the distribution of cardiac failure sign event in grayscale in accordance with the occurrence frequency of the events.

Note that the processing circuitry 11 may display only the last or latest event in a specific period during which events have occurred. Alternatively, the processing circuitry 11 may display only a portion, of a specific period during which events have occurred, in which events have concentrated most.

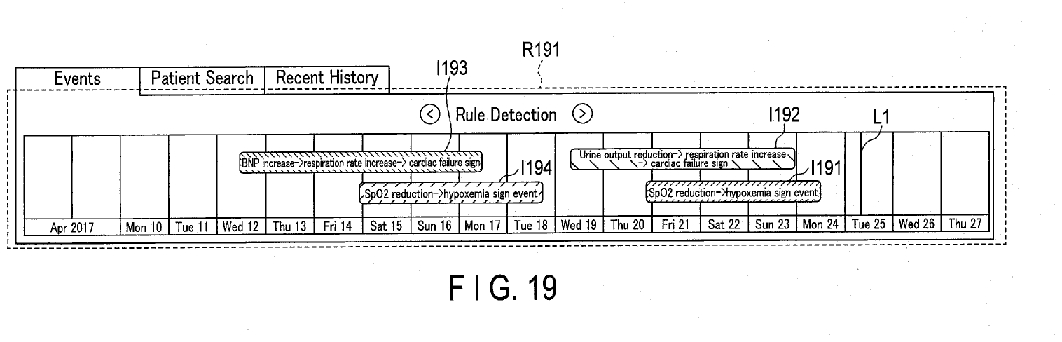

The last will describe a case in which, based on a combination of events that have occurred concerning a plurality of event types, the processing circuitry 11 summarizes and displays the events for each patient. In this case, the processing circuitry 11 can summarize events with proper granularity and indicate the similarity of the summarized events by classifying the occurrence patterns of the events using a technique such as clustering. FIG. 19 is a view showing another display example of mapping data according to the fourth embodiment. Referring to FIG. 19, the processing circuitry 11 summarizes and displays events of a plurality of event types that have occurred for each patient in consideration of the occurrence order of the events.

Referring to FIG. 19, a mapping area R191 includes icons I191, I192, I193, and I194 representing the respective patients. The respective icons included in the mapping area 8191 shown in FIG. 19 are displayed in different colors for each patient. The icon I191 represents, in a strip form, the fact that events have occurred, concerning, for example, the patient with patient ID “00000001”, in the order of event types “SpO2 reduction event” and “hypoxemia sign event” over a predetermined period. The icon I192 represents, in a strip form, the fact that events have occurred, concerning, for example, the patient with patient ID “00000002”, in the order of event types “urine output reduction event”, “respiration rate increase event”, and “cardiac failure sign event” over a predetermined period. The icon I193 represents, in a strip form, the fact that events have occurred, concerning, for example, the patient with patient ID “00000003”, in the order of event types “BNP increase event”, “respiration rate increase event”, and “cardiac failure sign event” over a predetermined period. The icon I194 represents, in a strip form, the fact that events have occurred, concerning, for example, the patient with patient ID “00000004”, in the order of event types “SpO2 reduction event” and “hypoxemia sign event” over a predetermined period.

Note that the mapping data shown in FIG. 19 is displayed such that similar combinations of events are set on the same row (at the same height).

### Other Embodiments

In the above embodiment, the processing circuitry 11 may change the contents of display concerning event associated information displayed in the event associated information area R42 in synchronism with, for example, a change in the time axis included in the mapping data represented by the mapping area R41 shown in FIG. 4. The following will describe a case in which the period indicated by the time axis included in the mapping data represented by the mapping area R41 shown in FIG. 4 is changed from “the period from Apr. 10, 2017 to Apr. 27, 2017” to “the period from Apr. 5, 2017 to Apr. 22, 2017”.

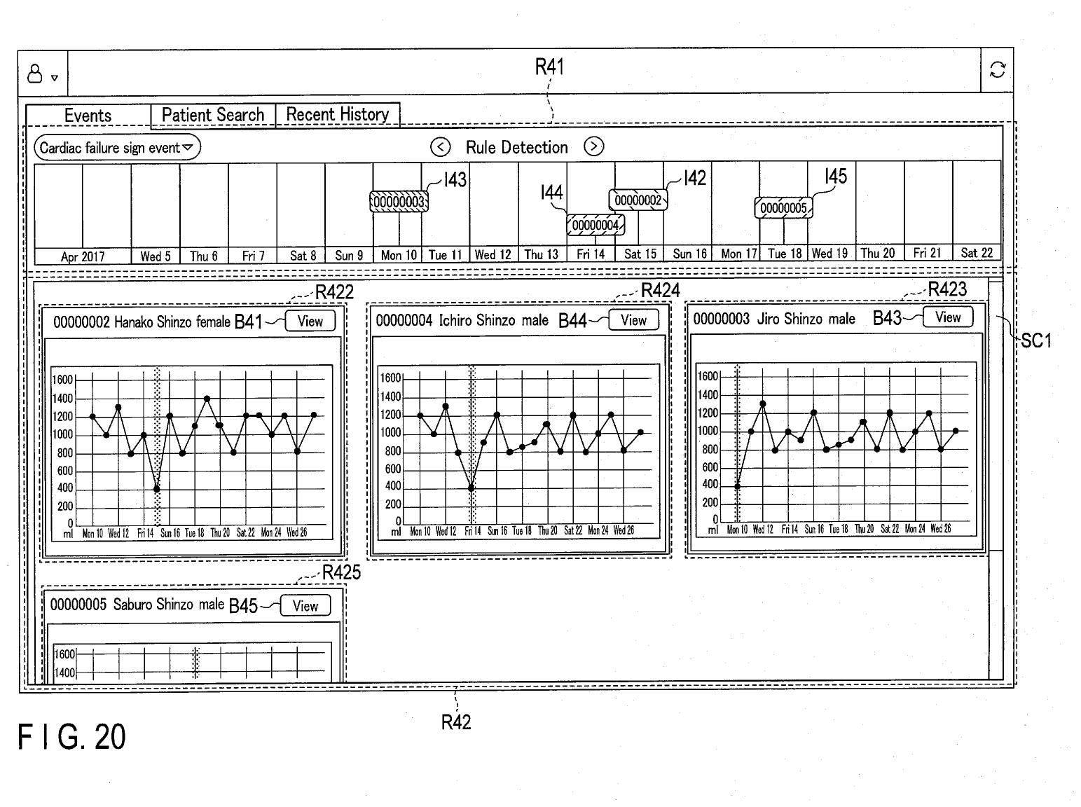

FIG. 20 is a view showing an example of a medical treatment screen according to another embodiment. FIG. 20 indicates a medical treatment screen after the range of the time axis included in the mapping data represented by the mapping area R41 shown in FIG. 4 is changed. Referring to FIG. 20, “the period from Apr. 23, 2017 to Apr. 27, 2017” represented by the time axis displayed in the mapping area R41 shown in FIG. 4 is hidden after the range of the time axis is changed. In this case, the icons I41a, I41b, and I41c are also hidden after the range of the time axis is changed. In addition, referring to FIG. 20, the area R421 corresponding to the icons I41a, I41b, and I41c representing patient ID “00000001” included in the event associated information area R42 shown in FIG. 4 is hidden after the range of the time axis is changed. In this manner, the processing circuitry 11 can change the contents of event associated information displayed in the event associated information area R42 in synchronism with a change in the range of the time axis. Note that the time axis represented by each event associated information included in the event associated information area R42 may be changed in interlocking with a change in the time axis included in the mapping data represented by the mapping area R41. The processing circuitry 11 may also optimize the contents of event associated information displayed in the event associated information area R42 in accordance with a patient.

In the above embodiment, for example, the processing circuitry 11 displays the information acquired concerning one patient in one graph area in the event associated information area R42 shown in FIG. 4. However, this is not exhaustive. The processing circuitry 11 may display the information concerning a plurality of patients in one graph area.

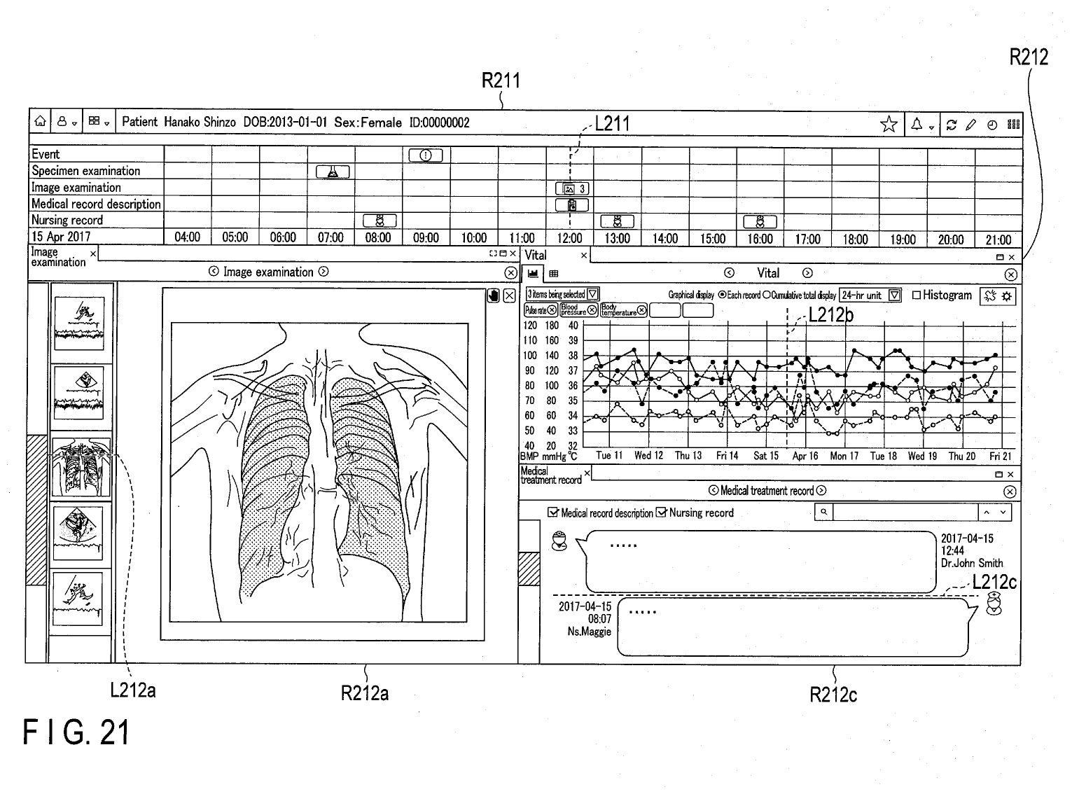

In the above embodiment, when, for example, each “View” button shown in FIG. 4 is designated, the processing circuitry 11 displays the details of medical treatment information of the corresponding patient. The following will describe a case in which the button B42 included in the area 422 included in the event associated information area R42 shown in FIG. 4 is designated. When the button B42 shown in FIG. 4 is designated, the processing circuitry 11 displays the details of the medical treatment information of the patient with patient ID “00000002”. FIG. 21 is a view showing an example of a patient detail screen indicating the details of the medical treatment information about the patient according to another embodiment. The patient detail screen shown in FIG. 21 indicates that the name, birth date, and sex of the patient with patient ID “00000002” are respectively “Hanako Shinzo”, “Jan. 1, 2013”, and “female”.

The patient detail screen shown in FIG. 21 includes a medical treatment information area divided into a timeline area R211, an image information area R212a displaying an image for an image examination, a vital sign information area R212b displaying vital sign information, and a medical treatment record information area R212c displaying the information of a medical treatment record.

The timeline area R211 displays a plurality of icons respectively corresponding to a plurality of pieces of medical treatment information in time series over the period from “04:00” to “21:00” on “Apr. 15, 2017”. The timeline area R211 includes an indicator L211 indicates the position of the date (designated date “12:00 on. Apr. 15, 2017”) designated by the operator.

The image information area R212a includes an information display area R212d displaying thumbnails of examination images concerning a specific patient over a certain period in a time-series order (for example, in descending order of dates of images from the top of the screen). The information display area R212d includes an indicator L212a indicating a position corresponding to a designated date. Subsequently, “a thumbnail of an examination image” will be simply referred to as “a thumbnail”. Note that a position corresponding to a designated date in the information display area R212d corresponds to, for example, the upper or lower portion of a thumbnail associated with a date closest to a designated date. This position may be located above a thumbnail.

The vital sign information area R212b displays vital sign information concerning a specific patient over the period “from Apr. 11, 2017 to Apr. 21, 2017”. The vital sign information area R212b includes an indicator L212b indicating a position corresponding to a designated date.

The medical treatment record information area R212c displays the comments made by a doctor and a nurse concerning a specific patient over a certain period in a time-series order (for example, in descending order of dates of comments from the top of the screen). The medical treatment record information area R212c includes an indicator L212c indicating a position corresponding to a designated date. Note that a position corresponding to a designated date in the medical treatment record information area corresponds to, for example, the upper or lower portion of a comment associated with a date closest to the designated date. In addition, this position may be located on a comment.

In summary, on the medical treatment screen shown in FIG. 21, first of all, in response to setting of the indicator L211, the indicator L212a, an indicator L212b, and the indicator L212c are automatically set (or displayed).

Referring to FIG. 21, the processing circuitry 11 changes the display contents of the patient detail screen shown in FIG. 21 in accordance with the event type of event that occurs. For example, the processing circuitry 11 changes the initial layout of the patient detail screen shown in FIG. 21 in accordance with the contents of an event that occurs. In addition, the processing circuitry 11 changes the display contents displayed in the medical treatment information area shown in FIG. 21 in accordance with the contents of an event that occurs. Note that, as shown in FIG. 21, the processing circuitry 11 displays the medical treatment screen displayed immediately after the designation of the button B42 shown in FIG. 4, the timeline are R211, the image information area R212a, the vital sign information area R212b, and the medical treatment record information area R212c such that median value of the time axis included in each area becomes an event date.

The term “processor” used in the above description, for example, a CPU (Central Processing Unit), a GPU (Graphics Processing Unit), or circuitry such as an ASIC (Application Specific Integrated Circuit), or a programmable logic device (for example, SPLD (Simple Programmable Logic Device), CPLD (Complex Programmable Logic Device), or FPGA (Field Programmable Logic Device)). The processor implements a function by reading out and executing a program saved in the storage circuitry. Note that each processor according to this embodiment described above may be formed as a single processor to implement its function by combining a plurality of independent circuits in addition of being formed as single circuitry for each processor. In addition, a plurality of constituent elements in FIGS. 1 and 2 may be integrated into a single processor to implement its function.

According to at least one of the embodiments described above, it is possible to shorten the time taken to access medical treatment information about a patient to be medically treated preferentially.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical information display apparatus comprising:
processing circuitry configured to:
acquire patient identification information for identifying a patient in whom an event as a determination index in a medical treatment has occurred and time information representing a time associated with the event from medical treatment information of a plurality of patients;
generate mapping data by mapping one or more icons associated with the event along a common time axis based on the plurality of pieces of patient identification information and the plurality of pieces of time information so as to make the icons identifiable for each patient; and
display the mapping data on a display.

2. The apparatus according to claim 1, wherein the processing circuitry is further configured to generate the mapping data by mapping the icons specifying the patient along the common time axis.

3. The apparatus according to claim 1, wherein the processing circuitry is further configured to generate the mapping data by mapping the icons specifying the event along the common time axis so as to make the patients identifiable.

4. The apparatus according to claim 1, wherein the processing circuitry is further configured to display event associated information associated with an event for each patient together with the mapping data.

5. The apparatus according to claim 4, wherein the processing circuitry is further configured to summarize and display the event associated information.

6. The apparatus according to claim 1, wherein the processing circuitry is further configured to display medical treatment information about the patient in detail in accordance with a predetermined display instruction.

7. The apparatus according to claim 1, wherein the processing circuitry is further configured to:
acquire the patient identification information and the time information concerning a plurality of event types representing types of the event;
generate the mapping data concerning the plurality of event types based on the patient identification information and the time information; and
change a display form of the mapping data displayed on the display in accordance with an occurrence pattern of the plurality of event types for each patient included in the mapping data.

8. The apparatus according to claim 1, wherein the processing circuitry is further configured to display the event that is browsed by a user and the event that is not browsed by the user so as to make the event discriminable.

9. The apparatus according to claim 4, wherein the processing circuitry is further configured to change contents of the event associated information in synchronism with a change in range of the common time axis.

10. The apparatus according to claim 6, wherein the processing circuitry is further configured to change display contents of details of medical treatment information about the patient in accordance with an event type representing a type of the event.

11. The apparatus according to claim 1, wherein the event represents an event in a medical treatment workflow.

12. The apparatus according to claim 1, wherein the event represents an event detected from the medical treatment information about the patient based on an empirical rule concerning a medical treatment.
